(19) United States
(12) Patent Application Publication
(10) Pub. No.: US 2023/0295109 A1
(43) Pub. Date: Sep. 21, 2023

(54) PYRIDINONE COMPOUNDS FOR THE TREATMENT OF AUTOIMMUNE DISEASE

(71) Applicant: F. Hoffmann-La Roche AG, Little Falls, NJ (US)

(72) Inventors: Dongdong CHEN, Shanghai (CN); Fabian DEY, Zürich (CH); Xin HONG, Shanghai (CN); Xuefei TAN, Shanghai (CN); Jiasu XU, Shanghai (CN); Wei ZHU, Shanghai (CN); Ge ZOU, Shanghai (CN)

(73) Assignee: Hoffmann-La Roche Inc., Little Falls, NJ (US)

(21) Appl. No.: 18/040,207

(22) PCT Filed: Aug. 2, 2021
(86) PCT No.: PCT/EP2021/071550

§ 371 Date: Feb. 1, 2023

(30) Foreign Application Priority Data
Aug. 4, 2020 (WO) PCT/CN2020/106791

(51) Int. Cl. C07D 401/04; C07D 519/00; C07D 487/04; C07D 471/04; C07D 401/14; C07D 498/10; C07D 413/14

(52) U.S. Cl. CPC C07D 401/04; C07D 401/14; C07D 413/14; C07D 471/04; C07D 487/04; C07D 498/10; C07D 519/00

(57) ABSTRACT

The present invention relates to compounds of formula (I), (I), wherein R¹, R², R³, R⁴ and A are as described herein, and their pharmaceutically acceptable salt thereof, and compositions including the compounds and methods of using the compounds.

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren’s syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long-term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721.). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7, 8, 9 is considered as a key node in a broad of autoimmune and auto-inflammatory diseases (Krieg, A. M. et al. Immunol. Rev. 2007, 220, 251. Jiménez-Dalmaroni, M. J. et al Autoimmun Rev. 2016, 15, 1. Chen, J. Q., et al. Clinical Reviews in Allergy & Immunology 2016, 50, 1.). Anti-RNA and anti-DNA antibodies are well-established diagnostic markers of SLE, and these antibodies can deliver both self-RNA and self-DNA to endosomes. While self-RNA complexes can be recognized by TLR7 and TLR8, self-DNA complexes can trigger TLR9 activation. Indeed, defective clearance of self-RNA and self-DNA from blood and/or tissues is evident in SLE (Systemic Lupus Erythematosus) patients. TLR7 and TLR9 have been reported to be upregulated in SLE tissues, and correlate with chronicity and activity of lupus nephritis, respectively. In B cells of SLE patients, TLR7 expression correlates with anti-RNP antibody production, while TLR9 expression with IL-6 and anti-dsDNA antibody levels. Consistently, in lupus mouse models, TLR7 is required for anti-RNA antibodies, and TLR9 is required for anti-nucleosome antibody. On the other hand, overexpression of TLR7 or human TLR8 in mice promotes autoimmunity and autoinflammation. Moreover, activation of TLR8 specifically contributes to inflammatory cytokine secretion of mDC/macrophages, neutrophil NETosis, induction of Th17 cells, and suppression of Treg cells. In addition to the described role of TLR9 in promoting autoantibody production of B cells, activation of TLR9 by self-DNA in pDC also leads to induction of type I IFNs and other inflammatory cytokines. Given these roles of TLR9 in both pDC and B cells, both as key contributors to the pathogenesis of autoimmune diseases, and the extensive presence of self-DNA complexes that could readily activate TLR9 in many patients with autoimmune diseases, it may have extra benefit to further block self-DNA mediated TLR9 pathways on top of inhibition of TLR7 and TLR8 pathways. Taken together, TLR7, 8 and 9 pathways represent new therapeutic targets for the treatment of autoimmune and auto-inflammatory diseases, for which no effective steroid-free and non-cytotoxic oral drugs exist, and inhibition of all these pathways from the very upstream may deliver satisfying therapeutic effects. As such, we invented oral compounds that target and suppress TLR7, TLR8 and TLR9 for the treatment of autoimmune and auto-inflammatory diseases.

### SUMMARY OF THE INVENTION

The present invention relates to novel compounds of formula (I),

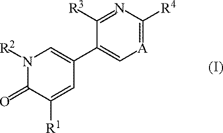

wherein
R¹ is C₁₋₆alkyl;
R² is C₁₋₆alkyl;
R³ is C₁₋₆alkyl or haloC₁₋₆alkyl;
R⁴ is piperazinyl, piperidinyl or 3,4,6,7,9,9a-hexahydro-1H-pyrazino[1,2-a]pyrazinyl, said piperazinyl, piperidinyl or 3,4,6,7,9,9a-hexahydro-1H-pyrazino[1,2-a]pyrazinyl being substituted by substituent selected from
   5,6,7,8-tetrahydro-1,6-naphthyridin-2-yl;
   phenylC₁₋₆alkyl, wherein phenyl is substituted by piperazinyl;
   piperazinyl;
   pyrazinylC₁₋₆alkyl, wherein pyrazinyl is substituted by piperazinyl;
   pyridinyl, wherein pyridinyl is substituted by piperazinyl;
   pyridinylC₁₋₆alkyl, wherein pyridinyl is substituted by 5-oxa-2,8-diazaspiro[3.5]nonan-2-yl or piperazinyl;
   pyrimidinyl, where pyrimidinyl is substituted by 5-oxa-2,8-diazaspiro[3.5]nonan-2-yl; and
   pyrimidinylC₁₋₆alkyl, wherein pyrimidinyl is substituted by amino(C₁₋₆alkyl)azetidinyl; 5-oxa-2,8-diazaspiro[3.5]nonan-2-yl; amino-1,4-oxazepan-4-yl or piperazinyl;
A is CH or N;
or a pharmaceutically acceptable salt thereof.

Another object of the present invention is related to novel compounds of formula (I). Their manufacture, medicaments based on a compound in accordance with the invention and their production as well as the use of compounds of formula (I) as TLR7 and TLR8 and TLR9 antagonist, and for the treatment or prophylaxis of systemic lupus erythematosus or lupus nephritis. The compounds of formula (I) show superior TLR7 and TLR8 and TLR9 antagonism activity. In addition, the compounds of formula (I) also show good cytotoxicity, phototoxicity, solubility, hPBMC, human microsome stability, AO (human cytosolic aldehyde oxidase) and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, trifluoroethyl, fluoromethyl, difluoromethyl, difluoroethyl or trifluoromethyl.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Antagonist of TLR7 and TLR8 and TLR9

The present invention relates to (i) a compound of formula (I),

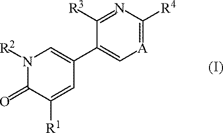

wherein
R¹ is C₁₋₆alkyl;
R² is C₁₋₆alkyl;
R³ is C₁₋₆alkyl or haloC₁₋₆alkyl;
R⁴ is piperazinyl, piperidinyl or 3,4,6,7,9,9a-hexahydro-1H-pyrazino[1,2-a]pyrazinyl, said piperazinyl, piperidinyl or 3,4,6,7,9,9a-hexahydro-1H-pyrazino[1,2-a]pyrazinyl being substituted by substituent selected from
   5,6,7,8-tetrahydro-1,6-naphthyridin-2-yl;
   phenylC₁₋₆alkyl, wherein phenyl is substituted by piperazinyl;
   piperazinyl;
   pyrazinylC₁₋₆alkyl, wherein pyrazinyl is substituted by piperazinyl;
   pyridinyl, wherein pyridinyl is substituted by piperazinyl;
   pyridinylC₁₋₆alkyl, wherein pyridinyl is substituted by 5-oxa-2,8-diazaspiro[3.5]nonan-2-yl or piperazinyl;
   pyrimidinyl, where pyrimidinyl is substituted by 5-oxa-2,8-diazaspiro[3.5]nonan-2-yl; and
   pyrimidinylC₁₋₆alkyl, wherein pyrimidinyl is substituted by amino(C₁₋₆alkyl)azetidinyl; 5-oxa-2,8-diazaspiro[3.5]nonan-2-yl; amino-1,4-oxazepan-4-yl or piperazinyl;
A is CH or N;
or a pharmaceutically acceptable salt thereof.

A further embodiment of present invention is (ii) a compound of formula (I) according to (i), or a pharmaceutically acceptable salt thereof, wherein A is CH.

A further embodiment of present invention is (iii) a compound of formula (I) according to (i) or (i), or a pharmaceutically acceptable salt thereof, wherein R⁴ is

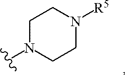

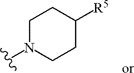

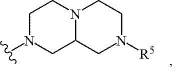

wherein R⁵ is selected from
5,6,7,8-tetrahydro-1,6-naphthyridin-2-yl;
phenylC₁₋₆alkyl, wherein phenyl is substituted by piperazinyl;
piperazinyl;
pyrazinylC₁₋₆alkyl, wherein pyrazinyl is substituted by piperazinyl;
pyridinyl, wherein pyridinyl is substituted by piperazinyl;
pyridinylC₁₋₆alkyl, wherein pyridinyl is substituted by 5-oxa-2,8-diazaspiro[3.5]nonan-2-yl or piperazinyl;
pyrimidinyl, where pyrimidinyl is substituted by 5-oxa-2,8-diazaspiro[3.5]nonan-2-yl; and
pyrimidinylC₁₋₆alkyl, wherein pyrimidinyl is substituted by amino(C₁₋₆alkyl)azetidinyl; 5-oxa-2,8-diazaspiro[3.5]nonan-2-yl; amino-1,4-oxazepan-4-yl or piperazinyl.

A further embodiment of present invention is (iv) a compound of formula (I), according to any one of (i) to (iii), or a pharmaceutically acceptable salt thereof, wherein R⁴ is

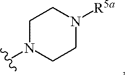

wherein R^{5a} is 5,6,7,8-tetrahydro-1,6-naphthyridin-2-yl, ((piperazinyl)phenyl)C₁₋₆alkyl, ((piperazinyl)pyrazinyl)C₁₋₆alkyl, ((piperazinyl)pyridinyl)C₁₋₆alkyl, ((5-oxa-2,8-diazaspiro[3.5]nonan-2-yl)pyridinyl)C₁₋₆alkyl, ((amino(C₁₋₆alkyl)azetidinyl)pyrimidinyl)C₁₋₆alkyl, ((5-oxa-2,8-diazaspiro[3.5]nonan-2-yl)pyrimidinyl)C₁₋₆alkyl, ((amino-1,4-oxazepan-4-yl)pyrimidinyl)C₁₋₆alkyl or ((piperazinyl)pyrimidinyl)C₁₋₆alkyl;

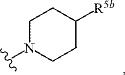

wherein R^{5b} is piperazinyl; or

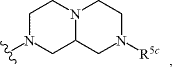

wherein R^{5c} is piperazinylpyridinyl or (5-oxa-2,8-diazaspiro[3.5]nonan-2-yl)pyrimidinyl.

A further embodiment of present invention is (v) a compound of formula (I) according to any one of (i) to (iv), wherein R⁴ is

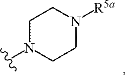

wherein R^{5a} is 5,6,7,8-tetrahydro-1,6-naphthyridin-2-yl, (4-piperazin-1-ylphenyl)methyl, (3-piperazin-1-ylphenyl)methyl, (5-piperazin-1-ylpyrazin-2-yl)methyl, (5-piperazin-1-yl-2-pyridinyl)methyl, (6-piperazin-1-yl-3-pyridinyl)methyl, [6-(5-oxa-2,8-diazaspiro[3.5]nonan-2-yl)-3-pyridinyl]methyl, [2-(3-amino-3-methyl-azetidin-1-yl)pyrimidin-5-yl]methyl, [2-(5-oxa-2,8-diazaspiro[3.5]nonan-2-yl)pyrimidin-5-yl]methyl, [2-[6-amino-1,4-oxazepan-4-yl]pyrimidin-5-yl]methyl, (2-piperazin-1-ylpyrimidin-5-yl)methyl or (5-piperazin-1-ylpyrimidin-2-yl)methyl;

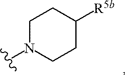

wherein R^{5b} is piperazin-1-yl; or

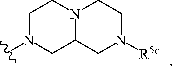

wherein R^{5c} is 6-piperazin-1-yl-3-pyridinyl or 2-(5-oxa-2,8-diazaspiro[3.5]nonan-2-yl)pyrimidin-5-yl.

A further embodiment of present invention is (vi) a compound of formula (I), or a pharmaceutically acceptable salt thereof, according to any one of (i) to (v), wherein R³ is C₁₋₆alkyl.

A further embodiment of present invention is (vii) a compound of formula (I), or a pharmaceutically acceptable salt thereof, according to any one of (i) to (vi), wherein R³ is ethyl or isopropyl.

A further embodiment of present invention is (viii) a compound of formula (I), or a pharmaceutically acceptable salt thereof, according to any one of (i) to (vii), wherein R⁴ is

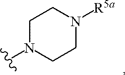

wherein R^{5a} is ((5-oxa-2,8-diazaspiro[3.5]nonan-2-yl)pyrimidinyl)C₁₋₆alkyl or ((amino-1,4-oxazepan-4-yl)pyrimidinyl)C₁₋₆alkyl;or

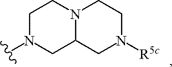

wherein R^{5c} is (5-oxa-2,8-diazaspiro[3.5]nonan-2-yl)pyrimidinyl.

A further embodiment of present invention is (ix) a compound of formula (I), or a pharmaceutically acceptable salt thereof, according to any one of (i) to (viii), wherein R⁴ is

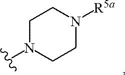

wherein R^{5a} is [2-(5-oxa-2,8-diazaspiro[3.5]nonan-2-yl)pyrimidin-5-yl]methyl or [2-(3-amino-3-methyl-azetidin-1-yl)pyrimidin-5-yl]methyl;

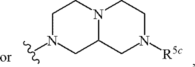

wherein R^{5c} is 2-(5-oxa-2,8-diazaspiro[3.5]nonan-2-yl)pyrimidin-5-yl.

A further embodiment of present invention is (x) a compound of formula (I), or a pharmaceutically acceptable salt thereof, according to any one of (i) to (ix), wherein
R¹ is C₁₋₆alkyl;
R² is C₁₋₆alkyl;
R³ is C₁₋₆alkyl;
R⁴ is

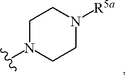

wherein R^{5a} is ((5-oxa-2,8-diazaspiro[3.5]nonan-2-yl)pyrimidinyl)C₁₋₆alkyl or ((amino-1,4-oxazepan-4-yl)pyrimidinyl)C₁₋₆alkyl; or

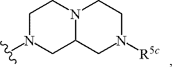

wherein R^{5c} is (5-oxa-2,8-diazaspiro[3.5]nonan-2-yl)pyrimidinyl;
A is CH;
or a pharmaceutically acceptable salt thereof.

A further embodiment of present invention is (xi) a compound of formula (I), or a pharmaceutically acceptable salt thereof, according to any one of (i) to (x), wherein
R¹ is methyl;
R² is methyl;
R³ is ethyl or isopropyl;
R⁴ is

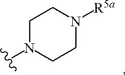

wherein R^{5a} is [2-(5-oxa-2,8-diazaspiro[3.5]nonan-2-yl)pyrimidin-5-yl]methyl or [2-(3-amino-3-methyl-azetidin-1-yl)pyrimidin-5-yl]methyl; or

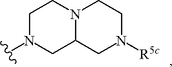

wherein R^{5c} is 2-(5-oxa-2,8-diazaspiro[3.5]nonan-2-yl)pyrimidin-5-yl;
A is CH;
or a pharmaceutically acceptable salt thereof.

Another embodiment of present invention is a compound of formula (I) selected from the following:
5-[2-ethyl-6-[4-[[2-(5-oxa-2,8-diazaspiro[3.5]nonan-2-yl)pyrimidin-5-yl]methyl]piperazin-1-yl]-3-pyridyl]-1,3-dimethyl-pyridin-2-one;
5-[2-ethyl-6-[4-[[6-(5-oxa-2,8-diazaspiro[3.5]nonan-2-yl)-3 pyridyl]methyl]piperazin-1-yl]-3-pyridyl]-1,3-dimethyl-pyridin-2-one;
5-[6-[4-[[2-(3-amino-3-methyl-azetidin-1-yl)pyrimidin-5-yl]methyl]piperazin-1-yl]-2-ethyl-3-pyridyl]-1,3-dimethyl-pyridin-2-one;
5-[6-[4-[[2-[(6S)-6-amino-1,4-oxazepan-4-yl]pyrimidin-5-yl]methyl]piperazin-1-yl]-2-ethyl-3-pyridyl]-1,3-dimethyl-pyridin-2-one;
5-[2-ethyl-6-[4-[(4-piperazin-1-ylphenyl)methyl]piperazin-1-yl]-3-pyridyl]-1,3-dimethyl-pyridin-2-one;
5-[2-isopropyl-6-[4-[[2-(5-oxa-2,8-diazaspiro[3.5]nonan-2-yl)pyrimidin-5-yl]methyl] piperazin-1-yl]-3-pyridyl]-1,3-dimethyl-pyridin-2-one;
5-[2-isopropyl-6-[4-[(5-piperazin-1-yl-2-pyridyl)methyl]piperazin-1-yl]-3-pyridyl]-1,3-dimethyl-pyridin-2-one;
5-[2-isopropyl-6-[4-[(5-piperazin-1-ylpyrazin-2-yl)methyl]piperazin-1-yl]-3-pyridyl]-1,3-dimethyl-pyridin-2-one;
5-[2-isopropyl-6-[4-[(2-piperazin-1-ylpyrimidin-5-yl)methyl]piperazin-1-yl]-3-pyridyl]-1,3-dimethyl-pyridin-2-one;
5-[6-[4-[[2-(3-amino-3-methyl-azetidin-1-yl)pyrimidin-5-yl]methyl]piperazin-1-yl]-2-isopropyl-3-pyridyl]-1,3-dimethyl-pyridin-2-one;
5-[2-isopropyl-6-[4-[(6-piperazin-1-yl-3-pyridyl)methyl]piperazin-1-yl]-3-pyridyl]-1,3-dimethyl-pyridin-2-one;
5-[2-isopropyl-6-[4-[(5-piperazin-1-ylpyrimidin-2-yl)methyl]piperazin-1-yl]-3-pyridyl]-1,3-dimethyl-pyridin-2-one;
5-[2-isopropyl-6-[4-[(4-piperazin-1-ylphenyl)methyl]piperazin-1-yl]-3-pyridyl]-1,3-dimethyl-pyridin-2-one;
5-[2-isopropyl-6-[4-[(3-piperazin-1-ylphenyl)methyl]piperazin-1-yl]-3-pyridyl]-1,3-dimethyl-pyridin-2-one;
5-[2-isopropyl-6-[4-(5,6,7,8-tetrahydro-1,6-naphthyridin-2-yl)piperazin-1-yl]-3-pyridyl]-1,3-dimethyl-pyridin-2-one;
5-[2-isopropyl-6-[8-(6-piperazin-1-yl-3-pyridyl)-3,4,6,7,9,9a-hexahydro-1H-pyrazino[1,2-a]pyrazin-2-yl]-3-pyridyl]-1,3-dimethyl-pyridin-2-one;
5-[2-(difluoromethyl)-6-[4-[(4-piperazin-1-ylphenyl)methyl]piperazin-1-yl]-3-pyridyl]-1,3-dimethyl-pyridin-2-one;
5-[2-isopropyl-6-(4-piperazin-1-yl-1-piperidyl)-3-pyridyl]-1,3-dimethyl-pyridin-2-one; and
5-[2-ethyl-6-[2-[2-(5-oxa-2,8-diazaspiro[3.5]nonan-2-yl)pyrimidin-5-yl]-3,4,6,7,9,9a-hexahydro-1H-pyrazino[1,2-a]pyrazin-8-yl]-3-pyridyl]-1,3-dimethyl-pyridin-2-one;
or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹, R², R³, R⁴ and A are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

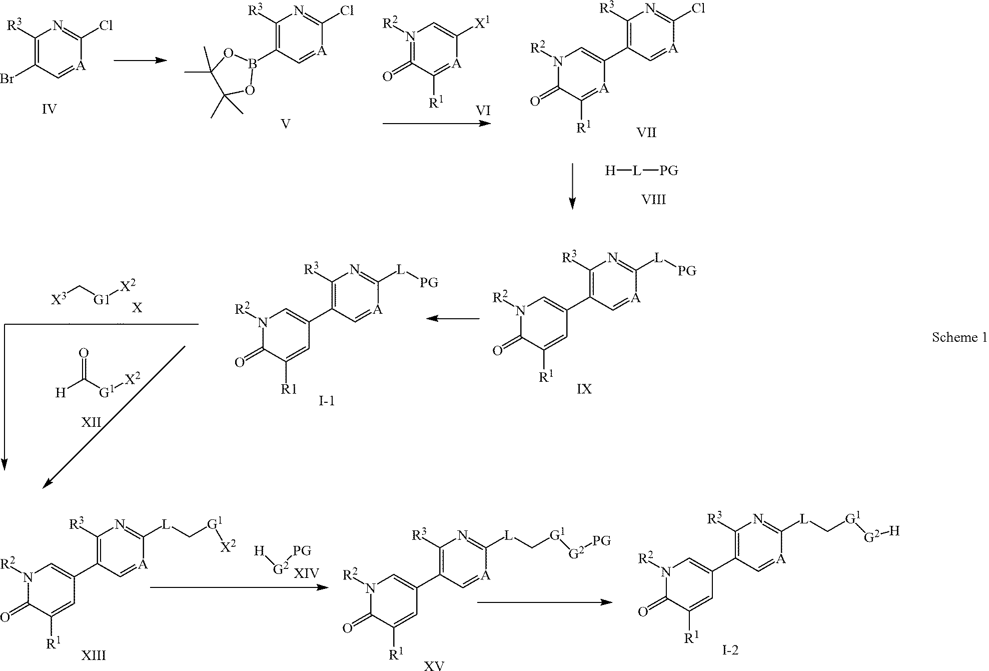

Wherein X¹, X², X³ are halogen; A is CH or N; PG is protecting group, such as Boc; L is piperazinyl, piperidinyl, piperazinylpiperidinyl, piperidinylpiperazinyl or 3,4,6,7,9,9a-hexahydro-1H-pyrazino[1,2-a]pyrazin-2-yl; G¹ is 5,6,7,8-tetrahydro-1,6-naphthyridin-2-yl, phenyl, piperazinyl, pyrazinyl, pyridinyl or pyrimidinyl; G² is piperazinyl, 5-oxa-2,8-diazaspiro[3.5]nonan-2-yl, amino(C₁₋₆alkyl)azetidinyl or amino-1,4-oxazepan-4-yl.

Compound of formula (IV) is treated with bis(pinacolato)diboron in the presence of a suitable base, such as KOAc, and a suitable palladium catalyst, such as PdCl₂(DPPF)-CH₂Cl₂ adduct, to afford compound of formula (V). Suzuki-coupling reaction between compound of formula (V) and compound of formula (VI) with a suitable catalyst, such as PdCl₂(DPPF)-CH₂Cl₂ adduct, and a suitable base, such as K₂CO₃, affords compound of formula (VII). Compound of formula (VII) undergoes Buchwald-Hartwig amination with compound (VIII) in the presence of a catalyst, such as RuPhos Pd G2, and a suitable base, such as Cs₂CO₃ or t-BuONa to afford compound of formula (IX). Deprotection of compound of formula (IX) under acidic condition, such as TFA, affords compound of (I-1). Substitution reaction between compound of formula (I-1) and compound of formula (X) in the presence of a suitable base, such as K₂CO₃, affords compound of formula (XIII). Compounds of formula (XIII) can also be obtained through reductive amination between compound of formula (X) and compound of formula (XII) with a reductant, such as NaBH(OAc)₃. Coupling of compound of formula (XIII) with compound of formula (XIV) under Buchwald-Hartwig amination conditions with a catalyst, such as RuPhos Pd G2, and a suitable base, such as Cs₂CO₃ or t-BuONa, affords compound of formula (XV). Deprotection of compound of formula (XV) under acidic condition, such as TFA, affords compound of (I-2).

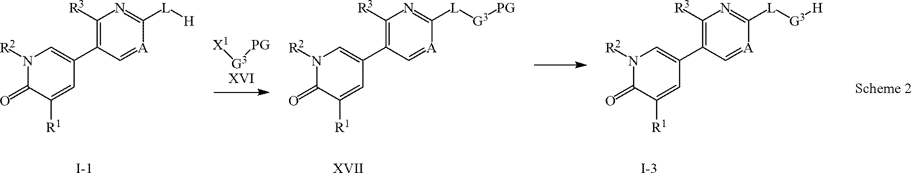

Wherein G³ is 5,6,7,8-tetrahydro-1,6-naphthyridin-2-yl.

Coupling of compound of formula (I-1) with compound of formula (XVI) under Buchwald-Hartwig amination conditions with a catalyst, such as RuPhos Pd G2, and a suitable base, such as Cs2CO3 or t-BuONa, affords compound of formula (XVII). Deprotection of compound of formula (XVII) under acidic condition, such as TFA, affords compound of formula (I-3).

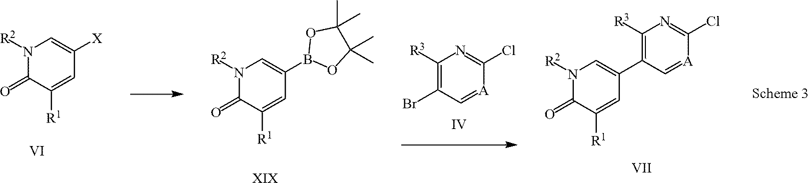

Compound of formula (VII) can also obtained via scheme 3.

Treating compound of formula (VI) with bis(pinacolato)diboron in the presence of a suitable base, such as KOAc, and a suitable palladium catalyst, such as PdCl₂(DPPF)-CH₂Cl₂ adduct, affords compound of formula (XIX). Coupling compound of formula (XIX) with compound of formula (IV) under Suzuki-coupling condition with a suitable catalyst, such as PdCl₂(DPPF)-CH₂Cl₂ adduct, and a suitable base, such as K₂CO₃, affords compound of formula (VII).

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising any of the following steps:
a) deprotection of compound of formula (IX),

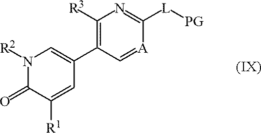

with an acid to afford compound of formula (I-1),

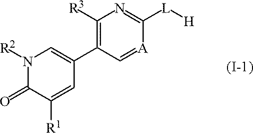

b) deprotection of compound of formula (XV),

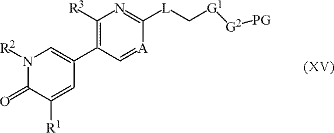

with an acid to afford compound of formula (I-2),

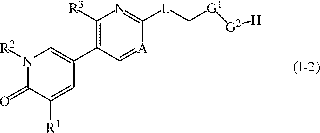

c) deprotection of compound of formula (XVII),

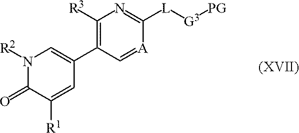

with an acid to afford compound of formula (I-3),

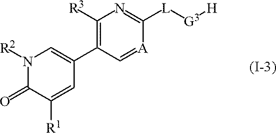

wherein
in step a),b) and c) the acid can be, for example, TFA;

A compound of formula (I) when manufactured according to the above process is also an object of the invention.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:

| | |
|---|---|
| ACN: | acetonitrile |
| Boc₂O: | di-tert butyl dicarbonate |
| CbzCl: | benzylchloroformate |
| DAST: | (Diethylamino)sulfur trifluoride |
| DEA: | diethylamine |
| DIPEA: | N,N-diisopropylethylamine |
| DMF: | N,N-dimethylformamide |
| EtOAc or EA: | ethyl acetate |
| FA: | formic acid |
| HLM | human liver microsome |
| IC₅₀: | half inhibition concentration |
| LCMS | liquid chromatography-mass spectrometry |
| MS: | mass spectrometry |
| PBS: | Phosphate Buffered Saline |
| [Pd(allyl)Cl]₂: | allylpalladium(II) chloride dimer |
| Pd[P(o-tol)₃]₂: | bis(tri-o-tolylphosphine)palladium |
| PE: | petroleum ether |
| prep-HPLC: | preparative high performance liquid chromatography |
| rt: | room temperature |
| RuPhos Pd G2: | chloro(2-dicyclohexylphosphino-2′,6′-diisopropoxy-1,1′-biphenyl)[2-(2′-amino-1,1′-biphenyl)]palladium(II) 2nd generation |
| PdCl₂(DPPF)-CH₂Cl₂ adduct: | [1,1′-Bis(diphenylphosphino)ferrocene]palladium(II) Dichloride Dichloromethane Adduct |
| DAST: | (Diethylamino)sulfur trifluoride |
| SFC: | supercritical fluid chromatography |
| TEA: | trimethylamine |
| TFA: | trifluoroacetic acid |

| | |
|---|---|
| v/v: | volume ratio |

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 µm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 µm, OBDTM 30 × 100 mm) column, SunFire™ Prep-C18 (5 µm, OBD™ 30 × 100 mm) column, Phenomenex Synergi-C18 (10 µm, 25 × 150 mm) or Phenomenex Gemini-C18 (10 µm, 25 × 150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 µm, 30 × 250 mm), AS (10 µm, 30 × 250 mm) or AD (10 µm, 30 × 250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

The following examples are intended to illustrate the meaning of the present invention but should by no means represent a limitation within the meaning of the present invention:

### Intermediate A1

### 3-BromoChloro-2-Ethylpyridine

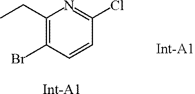

### Step 1: Preparation of 5-Bromo-6-Ethylpyridin-2-Amine

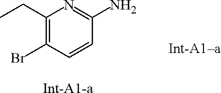

A mixture of 6-ethylpyridin-2-amine (5 g, 40.9 mmol, CAS No. 21717-29-3, vendor: Bide Pharmatech, catalog BD3776), 1-bromopyrrolidine-2,5-dione (8.01 g, 45 mmol, CAS No. 128-08-5, vendor: ALDRICH, catalog B81255) in MeOH (20 mL) was stirred at 0° C. for 16 hours. After the reaction was completed, the mixture was then concentrated in vacuo. The residue was then purified by flash column eluting with a gradient of MeOH/DCM (0% to 10%) to give 5-bromo-6-ethylpyridin-2-amine (4.1 g, 49.8% yield) as an orange solid. MS: calc’d 202 (M+H⁺), measured 202 (M+H⁺).

### Step 2: Preparation of 3-Bromo-6-Chloro-2-Ethylpyridine

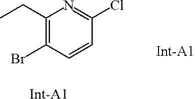

A mixture of 5-bromo-6-ethylpyridin-2-amine (4.1 g, 20.4 mmol), CuCl₂ (5.48 g, 40.8 mmol), tert-butyl nitrite (5.26 g, 51 mmol, CAS No. 540-80-7, vendor: TCI, catalog N0357) in DCM (40 mL) was stirred at 50° C. for 2 hours. After the reaction was completed, the mixture was then concentrated in vacuo. The residue was then purified by flash column eluting with a gradient of PE/EA (0% to 80%) to give 3-bromo-6-chloro-2-ethylpyridine (2.8 g, 62.3% yield) as yellow liquid. MS: calc’d 220 (M+H⁺), measured 220 (M+H⁺).

### Intermediate A2

### 6-Chloro-2-Ethyl-3-(4,4,5,5-Tetramethyl-1,3,2-Dioxaborolan-2-yl)Pyridine

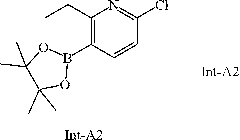

To a mixture of 3-bromo-6-chloro-2-ethylpyridine (2.8 g, 12.7 mmol), KOAc (3.12 g, 31.7 mmol), bis(pinacolato)diboron (3.55 g, 14 mmol, CAS No. 73183-34-3, vendor: Accela ChemBio Inc, catalog SY001323) in dioxane (20 mL) was added PdCl₂(DPPF)-CH₂Cl₂ adduct (929 mg, 1.27 mmol, CAS No. 95464-05-4, vendor: Accela ChemBio Inc, catalog SY002614) and the mixture was stirred at 90° C. under N₂ atmosphere for 2 hours. After the reaction was completed, the mixture was then concentrated in vacuo. The residue was then purified by flash column eluting with a gradient of PE/EA (0% to 10%) to give 6-chloro-2-ethyl-3-(4,4,5,5-tetramethyl-1,3,2-dioxaborolan-2-yl)pyridine (1.95 g, 57.4% yield) as a yellow solid. MS: calc’d 268 (M+H⁺), measured 268 (M+H⁺).

### Intermediate A3

### 3-BromoChloro-2-Isopropylpyridine

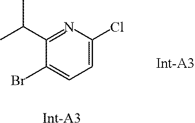

Int-A3 was prepared in analogy to the preparation of Int-A1 by using 6-isopropylpyridin-2-amine instead of 6-ethylpyridin-2-amine in step 1. MS calc’d 234 (M+H⁺), measured 234 (M+H⁺).

### Intermediate A4

### 6-Chloro-2-Isopropyl-3-(4,4,5,5-Tetramethyl-1,3,2-Dioxaborolan-2-yl)Pyridine

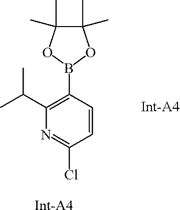

Int-A4 was prepared in analogy to the preparation of Int-A2 by using 3-bromo-6-chloro-2-isopropyl-pyridine instead of 3-bromo-6-chloro-2-ethylpyridine. MS calc’d 282 (M+H⁺), measured 282 (M+H⁺).

### Intermediate A5

### 3-BromoChloro-2-(Difluoromethyl)Pyridine

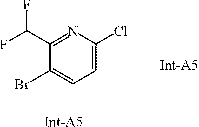

To a solution of 3-Bromo-6-chloropicolinaldehyde (1.5 g, 6.8 mmol, CAS No. 1060815-64-6, vendor: Bide Pharmatech, catalog BD259869) in DCM (40 mL) cooled at -78° C. was added DAST (4.39 g, 3.6 mL, 27.2 mmol, CAS No. 38078-09-0, vendor: PharmaBlock Sciences (Nanjing), Inc., catalog PBLY823 1). After addition, the mixture was stirred at -78° C. for another 30 minutes, then warmed to room temperature and stirred for 10 hours. After the reaction was completed, the mixture was concentrated in vacuo. The residue was then purified by flash column eluting with a gradient of PE/EA (0% to 10%) to give 3-bromo-6-chloro-2-(difluoromethyl)pyridine (1.5 g, 91% yield) as a yellow solid. MS: calc’d 242 (M+H⁺), measured 242 (M+H⁺).

### Intermediate B1

### 1,3-Dimethyl-5-(4,4,5,5-Tetramethyl-1,3,2-Dioxaborolan-2-yl)Pyridin-2-One

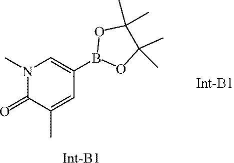

To a mixture of 5-bromo-1,3-dimethylpyridin-2(1H)-one (0.5 g, 2.47 mmol, CAS No. 51417-13-1, vendor: ALDRICH, catalog JRD0890), KOAc (291 mg, 2.97 mmol), bis(pinacolato)diboron (754 mg, 2.97 mmol) in the dioxane (10 mL) was added PdCl₂(DPPF)-CH₂Cl₂ adduct (90.5 mg, 124 µmol) and the mixture was stirred at 90° C. under N₂ atmosphere for 2 hours. After the reaction was completed, the mixture was then concentrated in vacuo. The residue was then purified by flash column eluting with a gradient of MeOH/DCM (0% to 10%) to give 1,3-dimethyl-5-(4,4,5,5-tetramethyl-1,3,2-dioxaborolan-2-yl)pyridin-2-one (616 mg, 100% yield) as a brown solid. MS: calc’d 250 (M+H⁺), measured 250 (M+H⁺).

### Example 1

### 5-Ethyl-6-[4-[(5-Oxa-2,8-Diazaspiro[3.5]Nonan-2-yl)Pyrimidin-5-yl]Methyl]Piperazin-1-yl]-3-Pyridyl]-1,3-Dimethyl-Pyridin-2-One

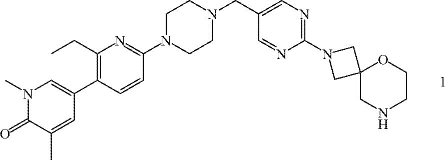 [SMILES: CCc1nc(N2CCN(Cc3cnc(N4CC5(CNCCO5)C4)nc3)CC2)ccc1-c1cc(C)c(=O)n(C)c1]

The title compound was prepared according to the following scheme

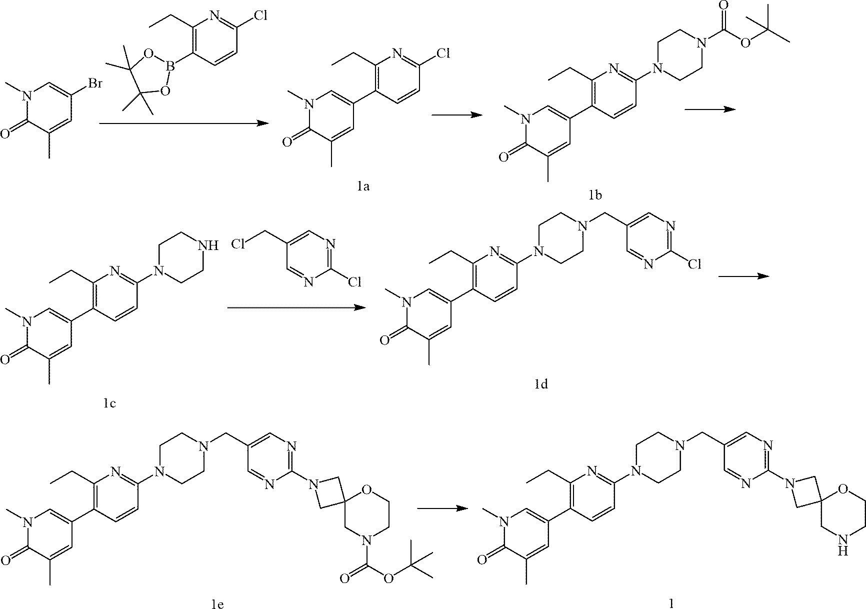

### Step 1: Preparation of 5-(6-Chloro-2-Ethyl-3-Pyridyl)-1,3-Dimethyl-Pyridin-2-One

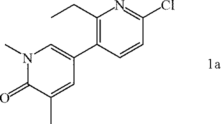 [SMILES: CCc1nc(Cl)ccc1-c1cc(C)c(=O)n(C)c1]

A mixture of 5-bromo-1,3-dimethylpyridin-2(1H)-one (238 mg, 1.19 µmol, CAS No. 51417-13-1, vendor: ALDRICH, catalog JRD0890), 6-chloro-2-ethyl-3-(4,4,5,5-tetramethyl-1,3,2-dioxaborolan-2-yl)pyridine (Int-A2, 318 mg, 1.19 mmol) and K₂CO₃ (205 mg, 1.48 mmol) in a mixed solvent of dioxane (5 mL) and water (1 mL) was added PdCl₂(DPPF)-CH₂Cl₂ adduct (72.4 mg, 99 µmol, CAS No. 95464-05-4, vendor: Accela ChemBio Inc, catalog SY002614) and the mixture was stirred at 80° C. under N₂ atmosphere for 2 hours. After the reaction was completed, the mixture was then concentrated in vacuo. The residue was then purified by flash column eluting with a gradient of MeOH/DCM (0% to 10%) to give 5-(6-chloro-2-ethyl-3-pyridyl)-1,3-dimethyl-pyridin-2-one (297 mg, 94.8% yield) as light brown oil. MS: calc’d 263 (M+H⁺), measured 263 (M+H⁺).

### Step 2: Preparation of Tert-Butyl 4-[5-(1,5-Dimethyl-6-Oxo-3-Pyridyl)-6-Ethyl-2-Pyridyl]Piperazine-1-Carboxylate

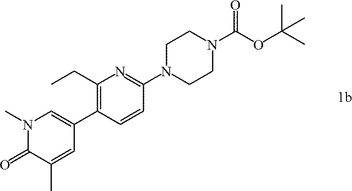 [SMILES: CCc1nc(N2CCN(C(=O)OC(C)(C)C)CC2)ccc1-c1cc(C)c(=O)n(C)c1]

To a mixture of 5-(6-chloro-2-ethyl-3-pyridyl)-1,3-dimethyl-pyridin-2-one (297 mg, 1.13 mmol), tert-butyl piperazine-1-carboxylate (274 mg, 1.47 mmol, CAS No. 77279-24-4, vendor: Bide Pharmatech, catalog B13517) and Cs₂CO₃ (552 mg, 1.7 mmol) in dioxane (5 mL) was added RuPhos Pd G2 (43.9 mg, 56.5 µmol, CAS No. 1375325-68-0, vendor: ALDRICH, catalog 753246) and the mixture was stirred at 110° C. under N₂ atmosphere for 16 hours. After the reaction was completed, the mixture was then concentrated in vacuo. The residue was then purified by flash column eluting with a gradient of MeOH/DCM (0% to 10%) to give tert-butyl 4-[5-(1,5-dimethyl-6-oxo-3-pyridyl)-6-ethyl-2-pyridyl]piperazine-1-carboxylate (287 mg, 61.5% yield) as orange oil. MS: calc’d 413 (M+H⁺), measured 413 (M+H⁺).

### Step 3: Preparation of 5-(2-Ethyl-6-Piperazin-1-yl-3-Pyridyl)-1,3-Dimethyl-Pyridin-2-One

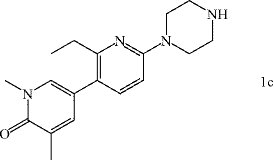 [SMILES: CCc1nc(N2CCNCC2)ccc1-c1cc(C)c(=O)n(C)c1]

To a solution of tert-butyl 4-[5-(1,5-dimethyl-6-oxo-3-pyridyl)-6-ethyl-2-pyridyl]piperazine-1-carboxylate (90 mg, 218 µmol) in DCM (4 mL) was added TFA (1 mL) and the mixture was then stirred at room temperature for 1 hour. After the reaction was completed, the mixture was then concentrated in vacuo. The residue was diluted with 2 M KOH solution (5 mL) and the resulting mixture was extracted with DCM (30 mL) twice. The combined organic layer was concentrated in vacuo to give the crude of 5-(2-ethyl-6-piperazin-1-yl-3-pyridyl)-1,3-dimethyl-pyridin-2-one (68 mg, 99.8% yield) as a yellow oil, which was usedin the next step directly without further purification. MS: calc’d 313 (M+H⁺), measured 313 (M+H⁺).

### Step 4: Preparation of 5-[6-[4-[(2-Chloropyrimidin-5-yl)Methyl]piperazin-1-yl]-2-Ethyl-3-Pyridyl]-1,3-Dimethyl-Pyridin-2-One

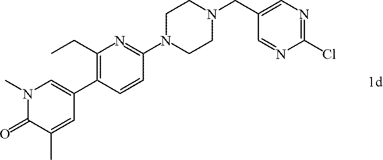 [SMILES: CCc1nc(N2CCN(Cc3cnc(Cl)nc3)CC2)ccc1-c1cc(C)c(=O)n(C)c1]

A mixture of 5-(2-ethyl-6-piperazin-1-yl-3-pyridyl)-1,3-dimethyl-pyridin-2-one (805 mg, 2.58 mmol), 2-chloro-5 (chloromethyl)-pyrimidine (2.1 g, 12.9 mmol, CAS No. 148406-13-7, vendor: PharmaBlock (Nanjing) R&D Co. Ltd, catalog PBN20120209) and K₂CO₃ (1.78 g, 12.9 mmol) in MeCN (10 mL) was stirred at 40° C. for 16 hours. After the reaction was completed, the mixture was filtered and the filtrate was concentrated in vacuo. The residue was then purified by flash column eluting with a gradient of MeOH/DCM (0% to 10%) to give 5-[6-[4-[(2-chloropyrimidin-5-yl)methyl]piperazin-1-yl]-2-ethyl-3-pyridyl]-1,3-dimethyl-pyridin-2-one (260 mg, 23% yield) as yellow oil. MS: calc’d 439 (M+H⁺), measured 439 (M+H⁺).

### Step 5: Preparation of Tert-Butyl 2-[5-[[4-[5-(1,5-Dimethyl-6-Oxo-3-Pyridyl)-6-Ethyl-2-Pyridyl]Piperazin-1-yl]Methyl]Pyrimidin-2-yl]-5-Oxa-2,8-Diazaspiro[3.5]Nonane-8-Carboxylate

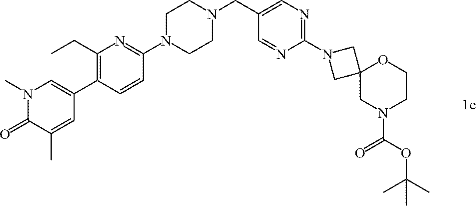 [SMILES: CCc1nc(N2CCN(Cc3cnc(N4CC5(CN(C(=O)OC(C)(C)C)CCO5)C4)nc3)CC2)ccc1-c1cc(C)c(=O)n(C)c1]

To a mixture of 5-[6-[4-[(2-chloropyrimidin-5-yl)methyl]piperazin-1-yl]-2-ethyl-3-pyridyl]-1,3-dimethyl-pyridin-2-one (65 mg, 148 µmol), tert-butyl 5-oxa-2,8-diazaspiro[3.5]nonane-8-carboxylate (67.6 mg, 296 µmol, CAS No. 1251011-05-8, vendor: PharmaBlock (Nanjing) R&D Co. Ltd, catalog PBN20111063) and Cs₂CO₃ (96.5 mg, 296 µmol) in dioxane (3 mL) was added Ruphos Pd G2 (5.75 mg, 7.4 µmol)and the mixture was stirred at 110° C. under N₂ atmosphere for 16 hours. After the reaction was completed, the mixture was then concentrated in vacuo. The residue was then purified by flash column eluting with a gradient of MeOH/DCM (0% to 10%) to give tert-butyl 2-[5-[[4-[5-(1,5-dimethyl-6-oxo-3-pyridyl)-6-ethyl-2-pyridyl]piperazin-1-yl]methyl]pyrimidin-2-yl]-5-oxa-2,8-diazaspiro[3.5]nonane-8-carboxylate (37 mg, 39.6% yield) as a yellow oil. MS: calc’d 631 (M+H⁺), measured 631 (M+H⁺).

### Step 6: Preparation of 5-[2-Ethyl-6-[4-[[2-(5-Oxa-2,8-Diazaspiro[3.5]Nonan-2-yl)Pyrimidin-5-yl]Methyl]Piperazin-1-yl]-3-Pyridyl]-1,3-Dimethyl-Pyridin-2-One

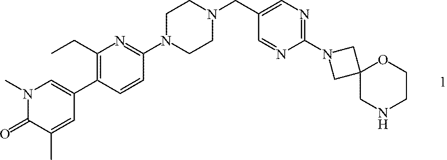 [SMILES: CCc1nc(N2CCN(Cc3cnc(N4CC5(CNCCO5)C4)nc3)CC2)ccc1-c1cc(C)c(=O)n(C)c1]

To a solution of tert-butyl 2-[5-[[4-[5-(1,5-dimethyl-6-oxo-3-pyridyl)-6-ethyl-2-pyridyl]piperazin-1-yl]methyl]pyrimidin-2-yl]-5-oxa-2,8-diazaspiro[3.5]nonane-8-carboxylate (37 mg, 58.7 µmol) in DCM (4 mL) was added TFA (1 mL) and the mixture was then stirred at room temperature for 1 hour. After the reaction was completed, the mixture was concentrated in vacuo and the residue was then purified by Prep-HPLC to give 5-[2-ethyl-6-[4-[[2-(5-oxa-2,8-diazaspiro[3.5]nonan-2-yl)pyrimidin-5-yl]methyl]piperazin-1-yl]-3-pyridyl]-1,3-dimethyl-pyridin-2-one (17 mg, 44.9% yield) as a white powder. ¹H NMR (400 MHz, CD₃OD, 298 K) δ (ppm) = 8.50 (s, 2H), 7.54 - 7.48 (m, 1H), 7.46 (d, J= 2.2 Hz, 1H), 7.36 (dd, J= 1.1, 2.3 Hz, 1H), 6.85 (d, J = 8.7 Hz, 1H), 4.35 - 4.29 (m, 2H), 4.28 - 4.23 (m, 2H), 4.16 - 4.11 (m, 2H), 4.11 -3.74 (m, 6H), 3.62 (s, 3H), 3.53 (s, 2H), 3.41 (br s, 4H), 3.29 - 3.23 (m, 2H), 2.71 (q, J = 7.5 Hz, 2H), 2.16 (s, 3H), 1.19 (t, J = 7.5 Hz, 3H). MS: calc’d 531 (M+H⁺), measured 531 (M+H⁺).

### Example 2

### 5-Ethyl-6-[4-[[6-(5-Oxa-2,8-Diazaspiro[3.5]Nonan-2-yl)-3 Pyridyl]Methyl]Piperazin-1-yl]-3-Pyridyl]-1,3-Dimethyl-Pyridin-2-One

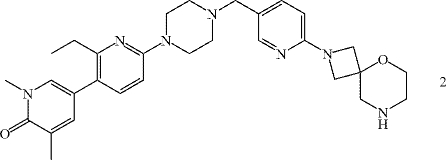 [SMILES: CCc1nc(N2CCN(Cc3ccc(N4CC5(CNCCO5)C4)nc3)CC2)ccc1-c1cc(C)c(=O)n(C)c1]

The title compound was prepared in analogy to the preparation of Example 1 by using 2-chloro-5-(chloromethyl)pyridine (CAS No. 70258-18-3, vendor: TCI) instead of 2-chloro-5-(chloromethyl)pyrimidine (CAS No. 148406-13-7, vendor: PharmaBlock (Nanjing) R&D Co. Ltd, catalog PBN20120209) in Step 4.

Example 2 (45 mg, 20.6%) was obtained as a white powder. ¹H NMR (400 MHz, CD₃OD, 298 K) δ (ppm) = 8.20 (d, J = 1.8 Hz, 1H), 7.90 (dd, J = 2.2, 8.9 Hz, 1H), 7.50 - 7.44 (m, 2H), 7.36 (dd, J = 1.1, 2.3 Hz, 1H), 6.87 - 6.80 (m, 1H), 6.77 (d, J= 8.9 Hz, 1H), 4.36 - 4.29 (m, 4H), 4.19 (d, J = 9.9 Hz, 2H), 4.05 - 3.75 (m, 6H), 3.65 - 3.60 (m, 3H), 3.59 - 3.54 (m, 2H), 3.44 -3.34 (m, 4H), 3.30 - 3.25 (m, 2H), 2.70 (q, J = 7.5 Hz, 2H), 2.16 (s, 3H), 1.19 (t, J = 7.5 Hz, 3H). MS: calc’d 530 (M+H⁺), measured 530 (M+H⁺).

### Example 3

### 5-[4-[[2-(3-Amino-3-Methyl-Azetidin-1-yl)Pyrimidin-5-yl]Methyl]Piperazin-1-yl]-2-Ethyl-3-Pyridyl]-1,3-Dimethyl-Pyridin-2-One

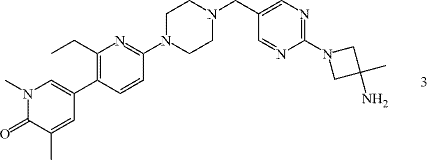 [SMILES: CCc1nc(N2CCN(Cc3cnc(N4CC(C)(N)C4)nc3)CC2)ccc1-c1cc(C)c(=O)n(C)c1]

The title compound was prepared in analogy to the preparation of Example 1 by using tert-butyl N-(3-methylazetidin-3-yl)carbamate (CAS No. 1018443-01-0, vendor: PharmaBlock (Nanjing) R&D Co. Ltd, catalog PB03046) instead of compound tert-butyl 5-oxa-2,8-diazaspiro[3.5]nonane-8-carboxylate (CAS No. 1251011-05-8, vendor: PharmaBlock (Nanjing) R&D Co. Ltd, catalog PBN20111063) in Step 5.

Example 3 (15 mg, 45.8%) was obtained as a yellow solid. ¹H NMR (400 MHz, CD₃OD, 298 K) δ (ppm) = 8.52 (s, 2H), 7.48 - 7.43 (m, 2H), 7.36 (dd, J = 1.1, 2.4 Hz, 1H), 6.82 (d, J = 8.7 Hz, 1H), 4.31 (s, 2H), 4.29 - 4.15 (m, 4H), 4.15 - 3.53 (m, 7H), 3.48 - 3.33 (m, 4H), 2.69 (q, J = 7.5 Hz, 2H), 2.16 (s, 3H), 1.69 (s, 3H), 1.19 (t, J = 7.5 Hz, 3H). MS: calc’d 489 (M+H⁺), measured 489 (M+H⁺).

### Example 4

### 5-[4-[[2-[(6S)-6-amino-1,4-oxazepan-4-yl]pyrimidin-5-yl]methyl]piperazin-1-yl]-2-ethyl-3-pyridyl]-1,3-dimethyl-pyridin-2-one

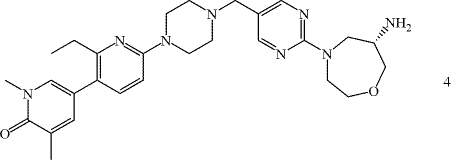 [SMILES: CCc1nc(N2CCN(Cc3cnc(N4CCOC[C@@H](N)C4)nc3)CC2)ccc1-c1cc(C)c(=O)n(C)c1]

The title compound was prepared in analogy to the preparation of Example 1 by using tert-butyl N-[(6S)-1,4-oxazepan-6-yl]carbamate (CAS No. 2306247-11-8, vendor: PharmaBlock (Nanjing) R&D Co. Ltd, catalog PB97931) instead of tert-butyl 5-oxa-2,8-diazaspiro[3.5]nonane-8-carboxylate (CAS No. 1251011-05-8, vendor: PharmaBlock (Nanjing) R&D Co. Ltd, catalog PBN20111063) in Step 5.

Example 4 (7 mg, 29.3%) was obtained as a yellow solid. ¹H NMR (400 MHz, CD₃OD, 298 K) δ (ppm) = 8.51 (s, 2H), 7.45-7.48 (m, 1H), 7.44 (d, J= 2.2 Hz, 1H), 7.34-7.37 (m, 1H), 6.79-6.84 (m, 1H), 4.43 (dd, J= 15.0, 4.6 Hz, 1H), 4.31 (s, 2H), 4.10-4.21 (m, 1H), 4.04 (s, 3H), 3.91 (br t, J = 2.9 Hz, 7H), 3.61 (s, 3H), 3.41 (br s, 4H), 2.69 (d, J = 7.6 Hz, 2H), 2.16 (s,3H), 1.19 ppm (t, J = 7.5 Hz, 3H). MS: calc’d 519 (M+H⁺), measured 519 (M+H⁺).

### Example 5

### 5-Ethyl-6-[4-[(4-Piperazin-1-Ylphenyl)Methyl]Piperazin-1-yl]-3-Pyridyl]-1,3-Dimethyl-Pyridin-2-One

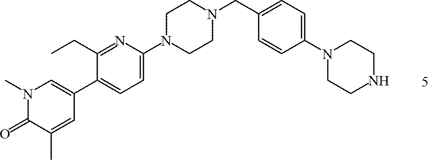 [SMILES: CCc1nc(N2CCN(Cc3ccc(N4CCNCC4)cc3)CC2)ccc1-c1cc(C)c(=O)n(C)c1]

The title compound was prepared according to the following scheme

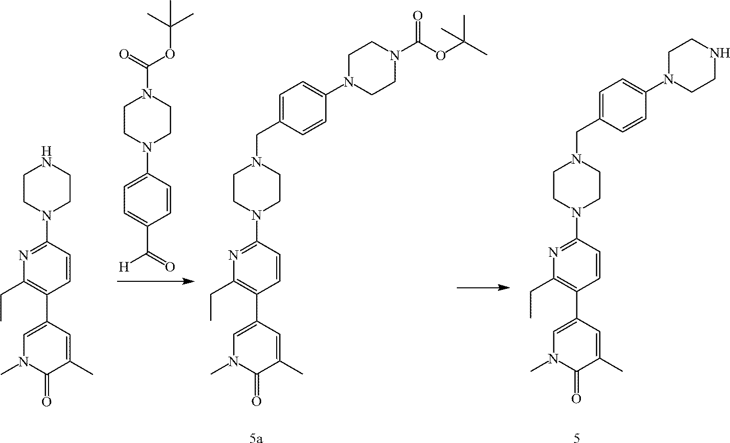

### Step 1: Preparation of Tert-Butyl 4-[4-[[4-[5-(1,5-Dimethyl-6-Oxo-3-Pyridyl)-6-Ethyl-2-Pyridyl]Piperazin-1-yl]Methyl]Phenyl]Piperazine-1-Carboxylate

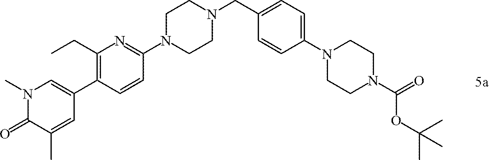 [SMILES: CCc1nc(N2CCN(Cc3ccc(N4CCN(C(=O)OC(C)(C)C)CC4)cc3)CC2)ccc1-c1cc(C)c(=O)n(C)c1]

A mixture of NaBH(OAc)₃ (369 mg, 1.74 mmol), 5-(2-ethyl-6-piperazin-1-yl-3-pyridyl)-1,3-dimethyl-pyridin-2-one (68 mg, 218 µmol), tert-butyl 4-(4-formylphenyl)piperazine-1-carboxylate (253 mg, 871 µmol, CAS No. 197638-83-8, vendor: Accela ChemBio, catalog SY031491) in DCM (10 mL) was stirred at room temperature for 16 hours. After the reaction was completed, the mixture was then concentrated in vacuo. The residue was then purified by flash column eluting with a gradient of MeOH/DCM (0% to 10%) to give tert-butyl 4-[4-[[4-[5-(1,5-dimethyl-6-oxo-3-pyridyl)-6-ethyl-2-pyridyl]piperazin-1-yl]methyl]phenyl]piperazine-1-carboxylate (19 mg, 14.9% yield) as a yellow oil. MS: calc’d 587 (M+H⁺), measured 587 (M+H⁺).

### Step 2: Preparation of 5-[2-Ethyl-6-[4-[(4-Piperazin-1-Ylphenyl)Methyl]Piperazin-1-yl]-3-Pyridyl]-1,3-Dimethyl-Pyridin-2-One

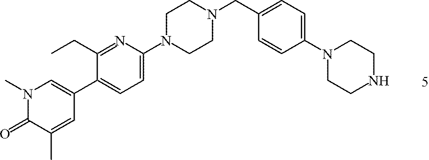 [SMILES: CCc1nc(N2CCN(Cc3ccc(N4CCNCC4)cc3)CC2)ccc1-c1cc(C)c(=O)n(C)c1]

To a solution of tert-butyl 4-[4-[[4-[5-(1,5-dimethyl-6-oxo-3-pyridyl)-6-ethyl-2-pyridyl]piperazin-1-yl]methyl]phenyl]piperazine-1-carboxylate in DCM (4 mL) was added TFA (1 mL) and the mixture stirred room temperature for 1 hour. After the reaction was completed, the mixture was concentrated in vacuo. The residue was then purified by Prep-HPLC to give 5-[2-ethyl-6-[4-[(4-piperazin-1-ylphenyl)methyl]piperazin-1-yl]-3-pyridyl]-1,3-dimethyl-pyridin-2-one (11 mg, 55.4% yield) as a yellow solid. ¹H NMR (400 MHz, CD₃OD, 298 K) δ (ppm) = 7.48 - 7.42 (m, 4H), 7.35 (d, J= 1.2 Hz, 1H), 7.13 (d, J= 8.8 Hz, 2H), 6.81 - 6.76 (m, 1H), 4.32 (s, 2H), 3.72 - 3.32 (m, 15H), 3.30 - 2.78 (m, 4H), 2.67 (q, J = 7.5 Hz, 2H), 2.16 (s, 3H), 1.18 (t, J = 7.5 Hz, 3H). MS: calc’d 487 (M+H⁺), measured 487 (M+H⁺).

### Example 6

### 5-Isopropyl-6-[4-[(5-Oxa-2,8-Diazaspiro[3.5]Nonan-2-yl)Pyrimidin-5-yl]Methyl] Piperazin-1-yl]-3-Pyridyl]-1,3-Dimethyl-Pyridin-2-One

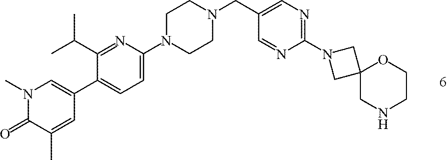 [SMILES: Cc1cc(-c2ccc(N3CCN(Cc4cnc(N5CC6(CNCCO6)C5)nc4)CC3)nc2C(C)C)cn(C)c1=O]

The title compound was prepared according to the following scheme

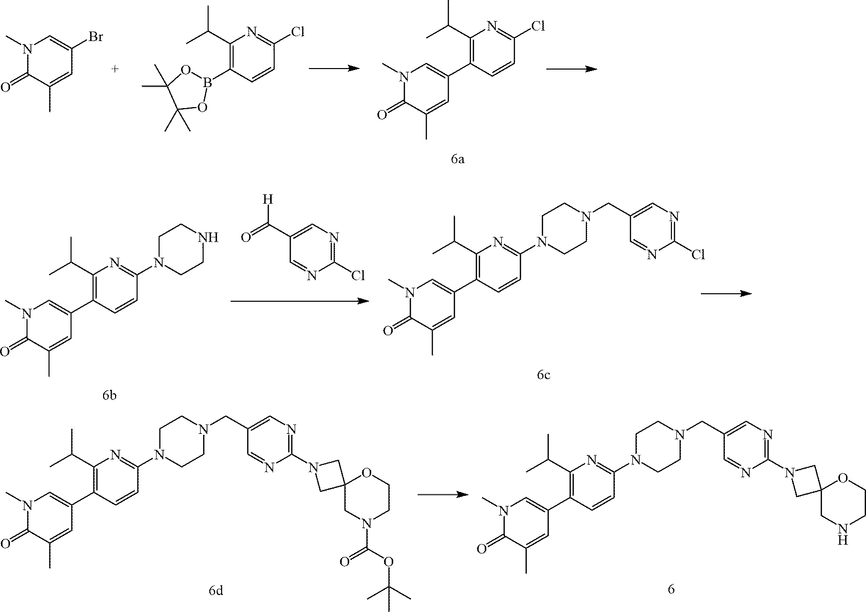

### Step1: Preparation of 5-(6-Chloro-2-Isopropyl-3-Pyridyl)-1,3-Dimethyl-Pyridin-2-One

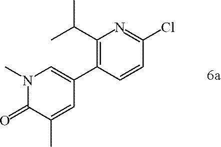 [SMILES: Cc1cc(-c2ccc(Cl)nc2C(C)C)cn(C)c1=O]

Compound 6a was prepared in analogy to the preparation of compound 1a by using 6-chloro-2-isopropyl-3-(4,4,5,5-tetramethyl-1,3,2-dioxaborolan-2-yl)pyridine (Int-4) instead of 6-chloro-2-ethyl-3-(4,4,5,5-tetramethyl-1,3,2-dioxaborolan-2-yl)pyridine in Step 1. MS: calc’d 277 (M+H⁺), measured 277 (M+H⁺).

### Step 2: Preparation 5-(2-Isopropyl-6-Piperazin-1-yl-3-Pyridyl)-1,3-Dimethyl-Pyridin-2-One

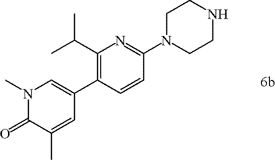 [SMILES: Cc1cc(-c2ccc(N3CCNCC3)nc2C(C)C)cn(C)c1=O]

Compound 6b was prepared in analogy to the preparation of compound 1c by using 5-(6-chloro-2-isopropyl-3-pyridyl)-1,3-dimethyl-pyridin-2-one instead of 5-(6-chloro-2-ethyl-3-pyridyl)-1,3-dimethyl-pyridin-2-one in Step 2. MS: calc’d 327 (M+H⁺), measured 327 (M+H⁺).

### Step 3: Preparation of 5-[6-[4-[(2-Chloropyrimidin-5-yl)Methyl]Piperazin-1-yl]-2-Isopropyl-3-Pyridyl]-1,3-Dimethyl-Pyridin-2-One

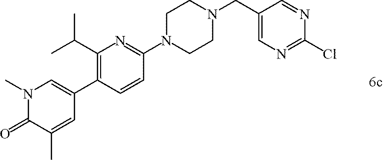 [SMILES: Cc1cc(-c2ccc(N3CCN(Cc4cnc(Cl)nc4)CC3)nc2C(C)C)cn(C)c1=O]

A mixture of 2-chloropyrimidine-5-carbaldehyde (180 mg, 1.26 mmol, CAS No. 933702-55-7, vendor: PharmaBlock Sciences (Nanjing), Inc., catalog PB01503), 5-(2-isopropyl-6-piperazin-1-yl-3-pyridyl)-1,3-dimethyl-pyridin-2-one (300 mg, 919 µmol), NaBH(OAc)₃ (300 mg, 1.42 mmol) in DCM (10 ml) was stirred at 25° C. for 16 hours. After the reaction was completed, the mixture was then concentrated in vacuo, the residue was then purified by flash column eluting with a gradient of MeOH/DCM (0% to 10%) to give 5-[6-[4-[(2-chloropyrimidin-5-yl)methyl]piperazin-1-yl]-2-isopropyl-3-pyridyl]-1,3-dimethyl-pyridin-2-one (460 mg, 110%) as a yellow oil. MS: calc’d 453 (M+H⁺), measured 453 (M+H⁺).

### Step 4: Preparation of 5-[2-Isopropyl-6-[4-[[2-(5-Oxa-2,8-Diazaspiro[3.5]Nonan-2-yl)Pyrimidin-5-yl]Methyl]Piperazin-1-yl]-3-Pyridyl]-1,3-Dimethyl-Pyridin-2-One

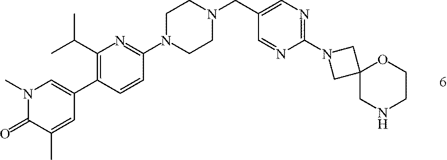 [SMILES: Cc1cc(-c2ccc(N3CCN(Cc4cnc(N5CC6(CNCCO6)C5)nc4)CC3)nc2C(C)C)cn(C)c1=O]

The title compound was prepared in analogy to the preparation of Example 1 by using 5-[6-[4-[(2-chloropyrimidin-5-yl)methyl]piperazin-1-yl]-2-isopropyl-3-pyridyl]-1,3-dimethyl-pyridin-2-one instead of 5-[6-[4-[(2-chloropyrimidin-5-yl)methyl]piperazin-1-yl]-2-ethyl-3-pyridyl]-1,3-dimethyl-pyridin-2-one in step 5.

Example 6 (30 mg) was obtained as a yellow solid. ¹H NMR (400 MHz, CD₃OD, 298 K) δ (ppm) = 8.50 (s, 2H), 7.43 - 7.38 (m, 2H), 7.34 - 7.31 (m, 1H), 6.76 (d, J = 8.6 Hz, 1H), 4.35 -4.21 (m, 5H), 4.13 (d, J = 10.1 Hz, 2H), 4.04 - 3.93 (m, 2H), 3.32 (br s, 10H), 3.30 - 3.03 (m, 5H), 2.16 (s, 3H), 1.18 (d, J = 6.7 Hz, 6H). MS: calc’d 545 (M+H⁺), measured 545 (M+H⁺).

### Example 7

### 5-Isopropyl-6-[4-[(5-Piperazin-1-yl-2-Pyridyl)Methyl]Piperazin-1-yl]-3-Pyridyl]-1,3-Dimethyl-Pyridin-2-One

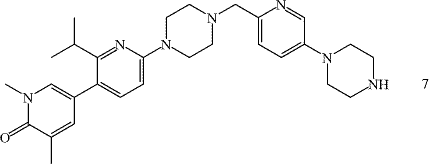 [SMILES: Cc1cc(-c2ccc(N3CCN(Cc4ccc(N5CCNCC5)cn4)CC3)nc2C(C)C)cn(C)c1=O]

### Step 1: Preparation of 5-[6-[4-[(5-Bromo-2-Pyridyl)Methyl]Piperazin-1-yl]-2-Isopropyl-3-Pyridyl]-1,3-Dimethyl-Pyridin-2-One

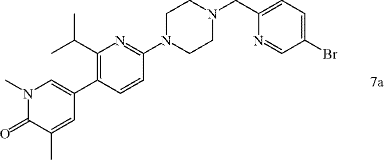 [SMILES: Cc1cc(-c2ccc(N3CCN(Cc4ccc(Br)cn4)CC3)nc2C(C)C)cn(C)c1=O]

Compound 7a was prepared in analogy to the preparation of compound 6c by using 5-bromopyridine-2-carbaldehyde instead of 2-chloropyrimidine-5-carbaldehyde. MS: calc’d 496 (M+H⁺), measured 496 (M+H⁺).

### Step 2: Preparation of 5-[2-Isopropyl-6-[4-[(5-Piperazin-1-yl-2-Pyridyl)Methyl]Piperazin-1-yl]-3-Pyridyl]-1,3-Dimethyl-Pyridin-2-One

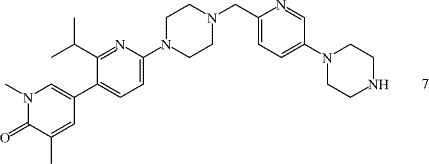 [SMILES: Cc1cc(-c2ccc(N3CCN(Cc4ccc(N5CCNCC5)cn4)CC3)nc2C(C)C)cn(C)c1=O]

The title compound was prepared in analogy to the preparation of Example 1 by using 5-[6-[4-[(5-bromo-2-pyridyl)methyl]piperazin-1-yl]-2-isopropyl-3-pyridyl]-1,3-dimethyl-pyridin-2-one and tert-butyl piperazine-1-carboxylate instead of 5-[6-[4-[(2-chloropyrimidin-5-yl)methyl]piperazin-1-yl]-2-ethyl-3-pyridyl]-1,3-dimethyl-pyridin-2-one and tert-butyl 5-oxa-2,8-diazaspiro[3.5]nonane-8-carboxylate in Step 5.

Example 7 (6.0 mg) was obtained as a yellow solid. ¹H NMR (400 MHz, CD₃OD, 298 K) δ (ppm) = 8.48 (d, J= 2.7 Hz, 1H), 7.55 - 7.50 (m, 1H), 7.48 - 7.44 (m, 1H), 7.43 - 7.38 (m, 2H), 7.34 - 7.31 (m, 1H), 6.76 (d, J = 8.7 Hz, 1H), 4.44 (s, 2H), 4.10 - 3.74 (m, 4H), 3.61 (s, 3H), 3.60 - 3.53 (m, 4H), 3.49 - 3.39 (m, 8H), 3.15 - 3.05 (m, 1H), 2.16 (s, 3H), 1.18 (d, J= 6.7 Hz, 6H). MS: calc’d 502 (M+H⁺), measured 502 (M+H⁺).

### Example 8

### 5-Isopropyl-6-[4-[(5-Piperazin-1-Ylpyrazin-2-yl)Methyl]piperazin-1-yl]-3-Pyridyl]-1,3-Dimethyl-Pyridin-2-One

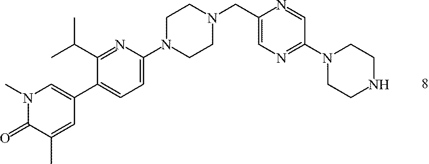 [SMILES: Cc1cc(-c2ccc(N3CCN(Cc4cnc(N5CCNCC5)cn4)CC3)nc2C(C)C)cn(C)c1=O]

### Step 1: Preparation of 5-[6-[4-[(5-Chloropyrazin-2-yl)Methyl]Piperazin-1-yl]-2-Isopropyl-3-Pyridyl]-1,3-Dimethyl-Pyridin-2-One

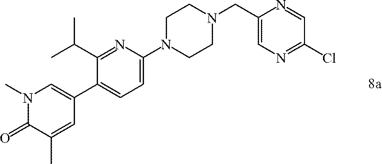 [SMILES: Cc1cc(-c2ccc(N3CCN(Cc4cnc(Cl)cn4)CC3)nc2C(C)C)cn(C)c1=O]

Compound 8a was prepared in analogy to the preparation of compound 1d by using 5-(2-isopropyl-6-piperazin-1-yl-3-pyridyl)-1,3-dimethyl-pyridin-2-one and 2-chloro-5-(chloromethyl)pyrazine (CAS No. 105985-21-5, vendor: Bide Pharmatech, catalog BD228124) instead of 5-(2-ethyl-6-piperazin-1-yl-3-pyridyl)-1,3-dimethyl-pyridin-2-one and 2-chloro-5-(chloromethyl)pyrimidine. MS: calc’d 453 (M+H⁺), measured 453 (M+H⁺).

### Step 2: Preparation of 5-[2-Isopropyl-6-[4-[(5-Piperazin-1-Ylpyrazin-2-yl)Methyl]Piperazin-1-yl]-3-Pyridyl]-1,3-Dimethyl-Pyridin-2-One

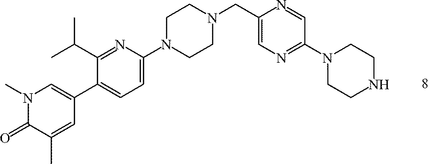 [SMILES: Cc1cc(-c2ccc(N3CCN(Cc4cnc(N5CCNCC5)cn4)CC3)nc2C(C)C)cn(C)c1=O]

The title compound was prepared in analogy to the preparation of Example 1 by using 5-[6-[4-[(5-chloropyrazin-2-yl)methyl]piperazin-1-yl]-2-isopropyl-3-pyridyl]-1,3-dimethyl-pyridin-2-one and tert-butyl piperazine-1-carboxylate instead of 5-[6-[4-[(2-chloropyrimidin-5-yl)methyl]piperazin-1-yl]-2-ethyl-3-pyridyl]-1,3-dimethyl-pyridin-2-one and tert-butyl 5-oxa-2,8-diazaspiro[3.5]nonane-8-carboxylate in Step 5.

Example 8 (26.0 mg) was obtained as a yellow solid. ¹H NMR (400 MHz, CD₃OD, 298 K) δ (ppm) = 8.54 - 8.40 (m, 1H), 8.28 (d, J = 1.3 Hz, 1H), 7.47 - 7.37 (m, 2H), 7.33 (dd, J= 1.0, 2.3 Hz, 1H), 6.76 (d, J = 8.7 Hz, 1H), 4.74 - 4.15 (m, 4H), 4.03 - 3.93 (m, 4H), 3.85 - 3.31 (m, 12H), 3.29 - 3.16 (m, 1H), 3.09 (qd, J = 6.7, 13.4 Hz, 1H), 2.16 (s, 3H), 1.18 (d, J = 6.6 Hz, 6H). MS: calc’d 503 (M+H⁺), measured 503 (M+H⁺).

### Example 9

### 5-Isopropyl-6-[4-[(2-Piperazin-1-Ylpyrimidin-5-yl)Methyl]piperazin-1-yl]-3-Pyridyl]-1,3-Dimethyl-Pyridin-2-One

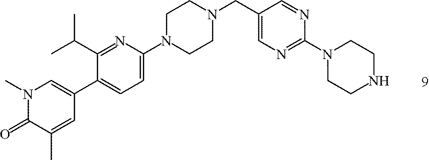 [SMILES: Cc1cc(-c2ccc(N3CCN(Cc4cnc(N5CCNCC5)nc4)CC3)nc2C(C)C)cn(C)c1=O]

The title compound was prepared in analogy to the preparation of Example 1 by using 5-[6-[4-[(2-chloropyrimidin-5-yl)methyl]piperazin-1-yl]-2-isopropyl-3-pyridyl]-1,3-dimethyl-pyridin-2-one and tert-butyl piperazine-1-carboxylate instead of 5-[6-[4-[(2-chloropyrimidin-5-yl)methyl]piperazin-1-yl]-2-ethyl-3-pyridyl]-1,3-dimethyl-pyridin-2-one and tert-butyl 5-oxa-2,8-diazaspiro[3.5]nonane-8-carboxylate in step 5.

Example 9 (28 mg, 51.6%) was obtained as a yellow solid. ¹H NMR (400 MHz, CD₃OD, 298 K) δ (ppm) = 8.57 - 8.52 (m, 2H), 7.43 - 7.39 (m, 2H), 7.34 - 7.31 (m, 1H), 6.76 (d, J = 8.6 Hz, 1H), 4.74 - 4.38 (m, 2H), 4.37 - 4.26 (m, 2H), 4.21 - 4.11 (m, 4H), 4.13 - 3.99 (m, 1H), 3.85 - 3.34 (m, 8H), 3.29 - 2.97 (m, 4H), 2.16 (s, 3H), 1.22 - 1.17 (m, 1H), 1.18 (d, J = 6.6 Hz, 6H). MS: calc’d 503 (M+H⁺), measured 503 (M+H⁺).

### Example 10

### 5-[4-[[2-(3-Amino-3-Methyl-Azetidin-1-yl)Pyrimidin-5-yl]Methyl]Piperazin-1-yl]-2-Isopropyl-3-Pyridyl]-1,3-Dimethyl-Pyridin-2-One

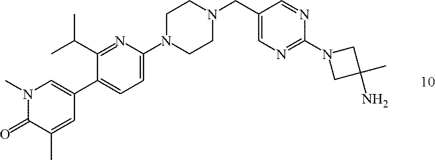

The title compound was prepared in analogy to the preparation of Example 1 by using 5-[6-[4-[(2-chloropyrimidin-5-yl)methyl]piperazin-1-yl]-2-isopropyl-3-pyridyl]-1,3-dimethyl-pyridin-2-one and tert-butyl N-(3-methylazetidin-3-yl)carbamate (CAS No. 1018443-01-0, vendor: PharmaBlock (Nanjing) R&D Co. Ltd, catalog PB03046) instead of 5-[6-[4-[(2-chloropyrimidin-5-yl)methyl]piperazin-1-yl]-2-ethyl-3-pyridyl]-1,3-dimethyl-pyridin-2-one and tert-butyl 5-oxa-2,8-diazaspiro[3.5]nonane-8-carboxylate in step 5.

Example 10 (28 mg, 42%) was obtained as an off-white powder. ¹H NMR (400 MHz, CD₃OD, 298 K) δ (ppm) = 8.52 (s, 2H), 7.43 - 7.38 (m, 2H), 7.32 (dd, J = 1.0, 2.3 Hz, 1H), 6.75 (d, J = 8.6 Hz, 1H), 4.44 - 4.01 (m, 7H), 3.89 - 3.32 (m, 8H), 3.29 - 3.02 (m, 2H), 2.16 (s, 3H), 1.70 (s, 3H), 1.18 (d, J = 6.7 Hz, 6H). MS: calc’d 503 (M+H⁺), measured 503 (M+H⁺).

### Example 11

### 5-Isopropyl-6-[4-[(6-Piperazin-1-yl-3-Pyridyl)Methyl]Piperazin-1-yl]-3-Pyridyl]-1,3-Dimethyl-Pyridin-2-One

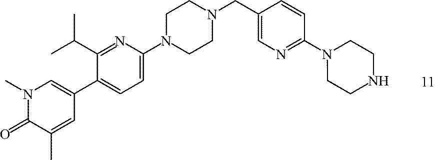 [SMILES: Cc1cc(-c2ccc(N3CCN(Cc4ccc(N5CCNCC5)nc4)CC3)nc2C(C)C)cn(C)c1=O]

### Step 1: Preparation of 5-[6-[4-[(6-Chloro-3-Pyridyl)Methyl]Piperazin-1-yl]-2-Isopropyl-3-Pyridyl]-1,3-Dimethyl-Pyridin-2-One

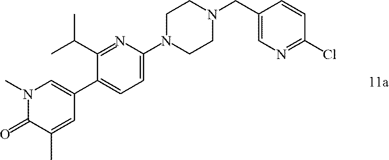 [SMILES: Cc1cc(-c2ccc(N3CCN(Cc4ccc(Cl)nc4)CC3)nc2C(C)C)cn(C)c1=O]

Compound 11a was prepared in analogy to the preparation of compound 1d by using 5-(2-isopropyl-6-piperazin-1-yl-3-pyridyl)-1,3-dimethyl-pyridin-2-one and 2-chloro-5-(chloromethyl)pyridine (CAS No. 70258-18-3, vendor: TCI) instead of 5-(2-ethyl-6-piperazin-1-yl-3-pyridyl)-1,3-dimethyl-pyridin-2-one and 2-chloro-5-(chloromethyl)pyrimidine. MS: calc’d 452 (M+H⁺), measured 452 (M+H⁺).

### Step 2: Preparation of 5-[2-Isopropyl-6-[4-[(6-Piperazin-1-yl-3-Pyridyl)Methyl]Piperazin-1-yl]-3-Pyridyl]-1,3-Dimethyl-Pyridin-2-One

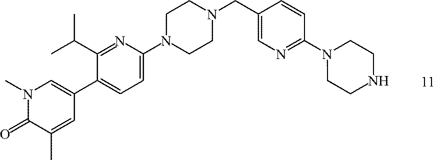 [SMILES: Cc1cc(-c2ccc(N3CCN(Cc4ccc(N5CCNCC5)nc4)CC3)nc2C(C)C)cn(C)c1=O]

The title compound was prepared in analogy to the preparation of Example 1 by using 5-[6-[4-[(6-chloro-3-pyridyl)methyl]piperazin-1-yl]-2-isopropyl-3-pyridyl]-1,3-dimethyl-pyridin-2-one and tert-butyl piperazine-1-carboxylate instead of 5-[6-[4-[(2-chloropyrimidin-5-yl)methyl]piperazin-1-yl]-2-ethyl-3-pyridyl]-1,3-dimethyl-pyridin-2-one and tert-butyl 5-oxa-2,8-diazaspiro[3.5]nonane-8-carboxylate in step 5.

Example 11 (28 mg, 53.7%) was obtained as a yellow solid. ¹H NMR (400 MHz, CD₃OD, 298 K) δ (ppm) = 8.30 (d, J = 2.3 Hz, 1H), 7.79 (dd, J = 2.4, 8.9 Hz, 1H), 7.43 - 7.38 (m, 2H), 7.34 - 7.31 (m, 1H), 7.04 (d, J = 8.9 Hz, 1H), 6.75 (d, J = 8.6 Hz, 1H), 4.83 - 4.07 (m, 4H), 4.01 -3.80 (m, 5H), 3.68 - 3.32 (m, 10H), 3.28 - 2.92 (m, 3H), 2.16 (s, 3H), 1.18 (d, J = 6.6 Hz, 6H). MS: calc’d 502 (M+H⁺), measured 502 (M+H⁺).

### Example 12

### 5-Isopropyl-6-[4-[(5-Piperazin-1-Ylpyrimidin-2-yl)Methyl]Piperazin-1-yl]-3-Pyridyl]-1,3-Dimethyl-Pyridin-2-One

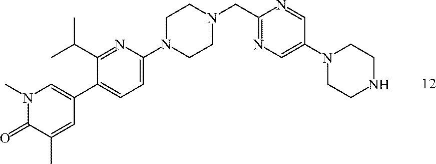 [SMILES: Cc1cc(-c2ccc(N3CCN(Cc4ncc(N5CCNCC5)cn4)CC3)nc2C(C)C)cn(C)c1=O]

### Step 1: Preparation of 5-[6-[4-[(5-Chloropyrimidin-2-yl)Methyl]Piperazin-1-yl]-2-Isopropyl-3-Pyridyl]-1,3-Dimethyl-Pyridin-2-One

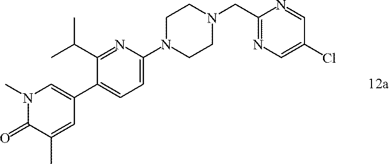 [SMILES: Cc1cc(-c2ccc(N3CCN(Cc4ncc(Cl)cn4)CC3)nc2C(C)C)cn(C)c1=O]

Compound 12a was prepared in analogy to the preparation of compound 6c by using 2-chloropyrimidine-5-carbaldehyde (CAS No. 933702-55-7, vendor: PharmaBlock Sciences (Nanjing), Inc., catalog PB01503) instead of 2-chloropyrimidine-5-carbaldehyde. MS: calc’d 453(M+H⁺), measured 453 (M+H⁺).

### Step 2: Preparation of 5-[2-Isopropyl-6-[4-[(5-Piperazin-1-Ylpyrimidin-2-yl)Methyl]piperazin-1-yl]-3-Pyridyl]-1,3-Dimethyl-Pyridin-2-One

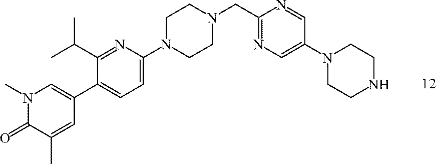 [SMILES: Cc1cc(-c2ccc(N3CCN(Cc4ncc(N5CCNCC5)cn4)CC3)nc2C(C)C)cn(C)c1=O]

The title compound was prepared in analogy to the preparation of Example 1 by using 5-[6-[4-[(5-chloropyrimidin-2-yl)methyl]piperazin-1-yl]-2-isopropyl-3-pyridyl]-1,3-dimethyl-pyridin-2-one and tert-butyl piperazine-1-carboxylate instead of 5-[6-[4-[(2-chloropyrimidin-5-yl)methyl]piperazin-1-yl]-2-ethyl-3-pyridyl]-1,3-dimethyl-pyridin-2-one and tert-butyl 5-oxa-2,8-diazaspiro[3.5]nonane-8-carboxylate in step 5.

Example 12 (30 mg, 42.5%) was obtained as a yellow solid. ¹H NMR (400 MHz, CD₃OD, 298 K) δ (ppm) = 8.63 (s, 2H), 7.44 - 7.39 (m, 2H), 7.35 - 7.32 (m, 1H), 6.78 (d, J = 8.7 Hz, 1H), 4.78 - 4.01 (m, 4H), 3.91 - 3.32 (m, 17H), 3.17 - 3.02 (m, 1H), 2.16 (s, 3H), 1.18 (d, J = 6.7 Hz, 6H). MS: calc’d 503 (M+H⁺), measured 503 (M+H⁺).

### Example 13

### 5-Isopropyl-6-[4-[(4-Piperazin-1-Ylphenyl)Methyl]Piperazin-1-yl]-3-Pyridyl]-1,3-Dimethyl-Pyridin-2-One

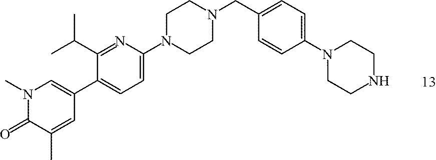 [SMILES: Cc1cc(-c2ccc(N3CCN(Cc4ccc(N5CCNCC5)cc4)CC3)nc2C(C)C)cn(C)c1=O]

The title compound was prepared in analogy to the preparation of Example 5 by using 5-(2-isopropyl-6-piperazin-1-yl-3-pyridyl)-1,3-dimethyl-pyridin-2-one instead of 5-(2-ethyl-6-piperazin-1-yl-3-pyridyl)-1,3-dimethyl-pyridin-2-one in Step 1.

Example 13 (28 mg, 43.3%) was obtained as a yellow solid. ¹H NMR (400 MHz, CD₃OD, 298 K) δ (ppm) = 7.45 (d, J = 8.8 Hz, 2H), 7.42 - 7.37 (m, 2H), 7.34 - 7.30 (m, 1H), 7.13 (d, J = 8.8 Hz, 2H), 6.74 (d, J = 8.6 Hz, 1H), 4.70 - 4.41 (m, 2H), 4.32 (s, 2H), 3.61 (s, 3H), 3.56 - 3.44 (m, 6H), 3.41 - 3.36 (m, 4H), 3.28 - 2.98 (m, 5H), 2.16 (s, 3H), 1.17 (d, J = 6.7 Hz, 6H). MS: calc’d 501 (M+H⁺), measured 501 (M+H⁺).

### Example 14

### 5-Isopropyl-6-[4-[(3-Piperazin-1-Ylphenyl)Methyl]Piperazin-1-yl]-3-Pyridyl]-1,3-Dimethyl-Pyridin-2-One

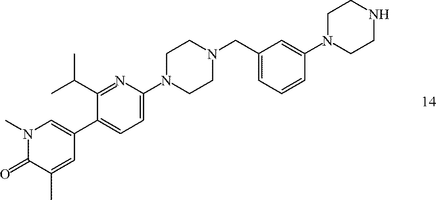 [SMILES: Cc1cc(-c2ccc(N3CCN(Cc4cccc(N5CCNCC5)c4)CC3)nc2C(C)C)cn(C)c1=O]

The title compound was prepared in analogy to the preparation of Example 5 by using 5-(2-isopropyl-6-piperazin-1-yl-3-pyridyl)-1,3-dimethyl-pyridin-2-one and tert-butyl 4-(3-formylphenyl)piperazine-1-carboxylate (CAS No. 1257849-25-4, vendor: Bide Pharmatech, catalog BD168751) instead of 5-(2-ethyl-6-piperazin-1-yl-3-pyridyl)-1,3-dimethyl-pyridin-2-one and tert-butyl 4-(4-formylphenyl)piperazine-1-carboxylate in Step 1.

Example 14 (39 mg, 66.7%) was obtained as a yellow solid. ¹H NMR (400 MHz, CD₃OD, 298 K) δ (ppm) = 7.65 - 7.59 (m, 1H), 7.58 - 7.51 (m, 1H), 7.45 - 7.31 (m, 5H), 6.73 (d, J= 8.6 Hz, 1H), 4.60 - 4.09 (m, 4H), 3.61 (s, 3H), 3.53 - 3.32 (m, 8H), 3.28 - 2.99 (m, 7H), 2.16 (s, 3H), 1.17 (d, J= 6.7 Hz, 6H). MS: calc’d 501 (M+H⁺), measured 501 (M+H⁺).

### Example 15

### 5-Isopropyl-6-[4-(5,6,7,8-Tetrahydro-1,6-Naphthyridin-2-yl)Piperazin-1-yl]-3-Pyridyl]-1,3-Dimethyl-Pyridin-2-One

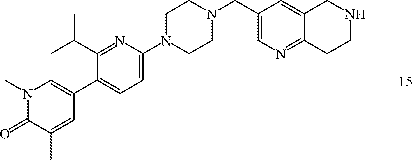 [SMILES: Cc1cc(-c2ccc(N3CCN(Cc4cnc5c(c4)CNCC5)CC3)nc2C(C)C)cn(C)c1=O]

The title compound was prepared according to the following scheme

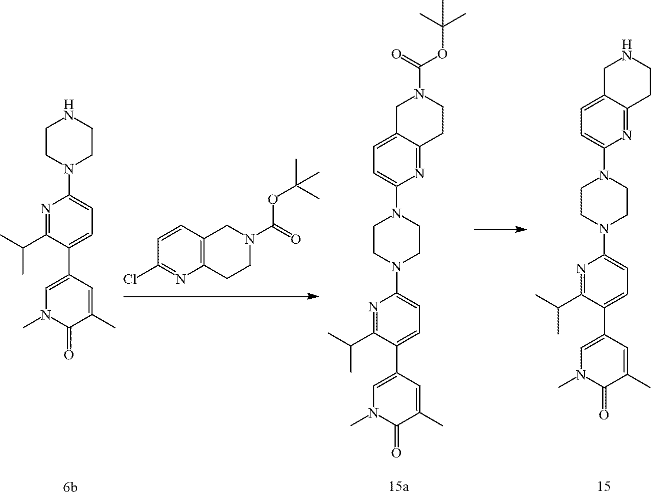

### Step 1: Preparation of Tert-Butyl 2-[4-[5-(1,5-Dimethyl-6-Oxo-3-Pyridyl)-6-Isopropyl-2-Pyridyl]Piperazin-1-yl]-7,8-Dihydro-SH-1,6-Naphthyridine-6-Carboxylate

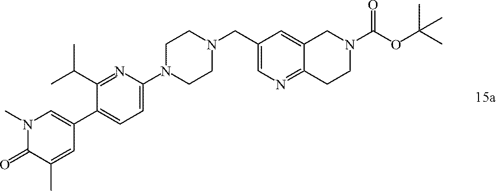 [SMILES: Cc1cc(-c2ccc(N3CCN(Cc4cnc5c(c4)CN(C(=O)OC(C)(C)C)CC5)CC3)nc2C(C)C)cn(C)c1=O]

To a mixture of 5-(2-isopropyl-6-piperazin-1-yl-3-pyridyl)-1,3-dimethyl-pyridin-2-one (100 mg, 306 µmol), tert-butyl 2-chloro-7,8-dihydro-5H-1,6-naphthyridine-6-carboxylate (206 mg, 766 µmol, CAS No. 1151665-15-4, vendor: Bide Pharmatech, catalog BD216990), Cs₂CO₃ (299 mg, 919 µmol)in dioxane (5 mL) was added RuPhos Pd G2 (47.6 mg, 61.3 µmol, CAS No. 1375325-68-0, vendor: ALDRICH, catalog 753246) and the mixture was stirred at 110° C. under N₂ atmosphere for 16 hours. After the reaction was completed, the mixture was then concentrated in vacuo, the residue was then purified by flash column eluting with a gradient of MeOH/DCM (0% to 10%) to give tert-butyl 2-[4-[5-(1,5-dimethyl-6-oxo-3-pyridyl)-6-isopropyl-2-pyridyl]piperazin-1-yl]-7,8-dihydro-SH-1,6-naphthyridine-6-carboxylate (100 mg, 58.4% yield) as yellow oil. MS: calc’d 559 (M+H⁺), measured 559 (M+H⁺).

### Step 2: Preparation of 5-[2-Isopropyl-6-[4-(5,6,7,8-Tetrahydro-1,6-Naphthyridin-2-yl)Piperazin-1-yl]-3-Pyridyl]-1,3-Dimethyl-Pyridin-2-One

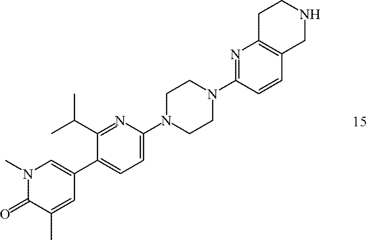 [SMILES: Cc1cc(-c2ccc(N3CCN(c4ccc5c(n4)CCNC5)CC3)nc2C(C)C)cn(C)c1=O]

To a solution of tert-butyl 2-[4-[5-(1,5-dimethyl-6-oxo-3-pyridyl)-6-isopropyl-2-pyridyl]piperazin-1-yl]-7,8-dihydro-5H-1,6-naphthyridine-6-carboxylate in DCM (4 mL) was added TFA (1 mL) and the mixture was then stirred at room temperature for 1 hour. After the reaction was completed, the mixture was concentrated in vacuo. The residue was then purified by Prep-HPLC to give 5-[2-isopropyl-6-[4-(5,6,7,8-tetrahydro-1,6-naphthyridin-2-yl)piperazin-1-yl]-3-pyridyl]-1,3-dimethyl-pyridin-2-one (34 mg) as a yellow solid. ¹H NMR (400 MHz, CD₃OD, 298 K) δ (ppm) = 7.66 (d, J = 9.2 Hz, 1H), 7.48 - 7.42 (m, 2H), 7.35 (dd, J = 1.0, 2.3 Hz, 1H), 7.11 (d, J = 9.2 Hz, 1H), 6.79 (d, J = 8.8 Hz, 1H), 4.29 (s, 2H), 3.89 - 3.79 (m, 8H), 3.63 - 3.61 (m, 3H), 3.21 - 3.15 (m, 2H), 3.15 - 3.07 (m, 1H), 2.16 (s, 3H), 2.03 (s, 2H), 1.23 (d, J = 6.7 Hz, 6H). MS: calc’d 459 (M+H⁺), measured 459 (M+H⁺).

### Example 16

### 5-Isopropyl-6-[8-(6-Piperazin-1-yl-3-Pyridyl)-3,4,6,7,9,9a-Hexahydro-1H-Pyrazino[1,2-a]Pyrazin-2-yl]-3-Pyridyl]-1,3-Dimethyl-Pyridin-2-One

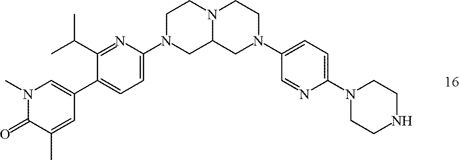 [SMILES: Cc1cc(-c2ccc(N3CCN4CCN(c5ccc(N6CCNCC6)nc5)CC4C3)nc2C(C)C)cn(C)c1=O]

The title compound was prepared according to the following scheme

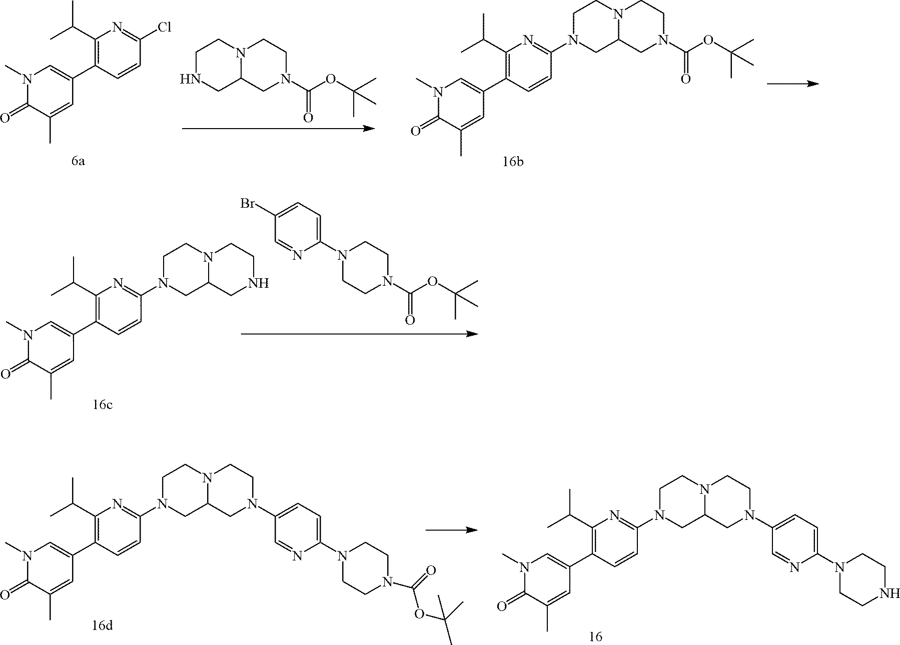

### Step 1: Preparation of Tert-Butyl 8-[5-(1,5-Dimethyl-6-Oxo-3-Pyridyl)-6-Isopropyl-2-Pyridyl]-3,4,6,7,9,9a-Hexahydro-1H-Pyrazino[1,2-a]Pyrazine-2-Carboxylate

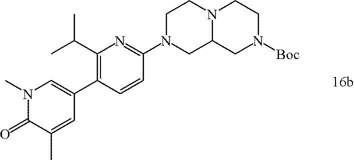 [SMILES: Cc1cc(-c2ccc(N3CCN4CCN(C(=O)OC(C)(C)C)CC4C3)nc2C(C)C)cn(C)c1=O]

To a mixture of 5-(6-chloro-2-isopropyl-3-pyridyl)-1,3-dimethyl-pyridin-2-one (100 mg, 361 µmol), tert-butyl 1,3,4,6,7,8,9,9a-octahydropyrazino[1,2-a]pyrazine-2-carboxylate (87.2 mg, 361 µmol, CAS No. 1159825-34-9, vendor: PharmaBlock Sciences (Nanjing), Inc., catalog PB07063) and Cs₂CO₃ (235 mg, 723 µmol) in dioxane (3 mL) was added RuPhos Pd G2 (14 mg, 18.1 µmol), the mixture was stirred at 110° C. under N₂ atmosphere for 16 hours. After the reaction was completed, the mixture was then concentrated in vacuo. The residue was then purified by flash column eluting with a gradient of MeOH/DCM (0% to 10%) to give tert-butyl 8-[5-(1,5-dimethyl-6-oxo-3-pyridyl)-6-isopropyl-2-pyridyl]-3,4,6,7,9,9a-hexahydro-1H-pyrazino[1,2-a]pyrazine-2-carboxylate (143 mg, 82.2% yield) as yellow oil. MS: calc’d 482 (M+H⁺), measured 482 (M+H⁺).

### Step 2: Preparation of 5-[6-(1,3,4,6,7,8,9,9a-Octahydropyrazino[1,2-a]Pyrazin-2-yl)-2-Isopropyl-3-Pyridyl]-1,3-Dimethyl-Pyridin-2-One

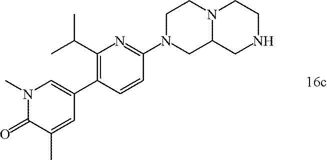 [SMILES: Cc1cc(-c2ccc(N3CCN4CCNCC4C3)nc2C(C)C)cn(C)c1=O]

To a solution of tert-butyl 8-[5-(1,5-dimethyl-6-oxo-3-pyridyl)-6-isopropyl-2-pyridyl]-3,4,6,7,9,9a-hexahydro-1H-pyrazino[1,2-a]pyrazine-2-carboxylate (143 mg, 297 µmol) in DCM (4 mL) was added TFA (1 mL) and the mixture was then stirred at room temperature for 1 hour. After the reaction was completed, the mixture was then concentrated in vacuo. The residue was diluted with 2 M KOH solution (5 mL) and the resulting mixture was extracted with DCM (30 mL) twice. The combined organic layer was concentrated in vacuo to give the crude of 5-[6-(1,3,4,6,7,8,9,9a-octahydropyrazino[1,2-a]pyrazin-2-yl)-2-isopropyl-3-pyridyl]-1,3-dimethyl-pyridin-2-one (87 mg, 76.8% yield) as a light brown oil, which was used in the next step directly without further purification. MS: calc’d 382 (M+H⁺), measured 382 (M+H⁺).

### Step 3: Preparation of Tert-Butyl 4-[5-[2-[5-(1,5-Dimethyl-6-Oxo-3-Pyridyl)-6-Isopropyl-2-Pyridyl]-3,4,6,7,9,9a-Hexahydro-1H-Pyrazino[1,2-a]Pyrazin-8-yl]-2-Pyridyl]Piperazine-1-Carboxylate

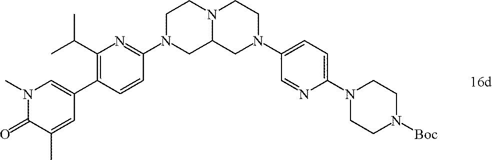 [SMILES: Cc1cc(-c2ccc(N3CCN4CCN(c5ccc(N6CCN(C(=O)OC(C)(C)C)CC6)nc5)CC4C3)nc2C(C)C)cn(C)c1=O]

To a mixture of 5-[6-(1,3,4,6,7,8,9,9a-octahydropyrazino[1,2-a]pyrazin-2-yl)-2-isopropyl-3-pyridyl]-1,3-dimethyl-pyridin-2-one (87 mg, 228 µmol), tert-butyl 4-(5-bromo-2-pyridyl)piperazine-1-carboxylate (156 mg, 456 µmol, CAS No. 153747-97-8, vendor: Accela ChemBio Inc, catalog SY101561) and Cs₂CO₃ (149 mg, 456 µmol)in dioxane (3 mL) was added RuPhos Pd G2 (8.86 mg, 11.4 µmol)and the mixture was stirred at 110° C. under N₂ atmosphere for 16 hours. After the reaction was completed, the mixture was concentrated in vacuo. The residue was then purified by flash column eluting with a gradient of MeOH/DCM (0% to 10%) to give tert-butyl 4-[5-[2-[5-(1,5-dimethyl-6-oxo-3-pyridyl)-6-isopropyl-2-pyridyl]-3,4,6,7,9,9a-hexahydro-1H-pyrazino[1,2-a]pyrazin-8-yl]-2-pyridyl]piperazine-1-carboxylate (16 mg, 10.9% yield) as a light brown oil. MS: calc’d 643 (M+H⁺), measured 643 (M+H⁺).

### Step 4: Preparation of 5-[2-Isopropyl-6-[8-(6-Piperazin-1-yl-3-Pyridyl)-3,4,6,7,9,9a-Hexahydro-1H-Pyrazino[1,2-a]Pyrazin-2-yl]-3-Pyridyl]-1,3-Dimethyl-Pyridin-2-One

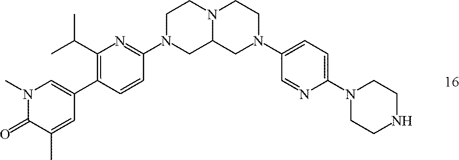 [SMILES: Cc1cc(-c2ccc(N3CCN4CCN(c5ccc(N6CCNCC6)nc5)CC4C3)nc2C(C)C)cn(C)c1=O]

To a solution of tert-butyl 4-[5-[2-[5-(1,5-dimethyl-6-oxo-3-pyridyl)-6-isopropyl-2-pyridyl]-3,4,6,7,9,9a-hexahydro-1H-pyrazino[1,2-a]pyrazin-8-yl]-2-pyridyl]piperazine-1-carboxylate (16 mg, 0.024 mmol) in DCM (4 mL) was added TFA (1 mL) and the mixture was then stirred at room temperature for 1 hour. After the reaction was completed, the mixture was concentrated in vacuo and the residue was then purified by Prep-HPLC to give 5-[2-isopropyl-6-[8-(6-piperazin-1-yl-3-pyridyl)-3,4,6,7,9,9a-hexahydro-1H-pyrazino[1,2-a]pyrazin-2-yl]-3-pyridyl]-1,3-dimethyl-pyridin-2-one (9 mg, 46.8% yield) as a yellow solid. ¹H NMR (400 MHz, CD₃OD, 298 K) δ (ppm) = 7.89 (d, J= 2.8 Hz, 1H), 7.79 (dd, J = 9.4, 2.9 Hz, 1H), 7.39-7.46 (m, 2H), 7.31-7.35 (m, 1H), 7.19 (d, J = 9.5 Hz, 1H), 6.81 (d, J = 8.7 Hz, 1H), 4.72 (br s, 2H), 3.91 (br d, J= 12.6 Hz, 1H), 3.58-3.71 (m, 6H), 3.19-3.46 (m, 12H), 2.99-3.19 (m, 3H), 2.16 (s, 3H), 1.16-1.23 (m, 7H). MS: calc’d 543 (M+H⁺), measured 543 (M+H⁺).

### Example 17

### 5-(Difluoromethyl)-6-[4-[(4-Piperazin-1-Ylphenyl)Methyl]Piperazin-1-yl]-3-Pyridyl]-1,3-Dimethyl-Pyridin-2-One

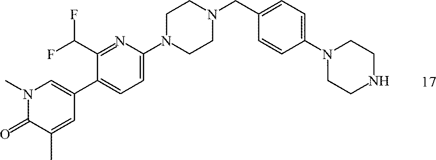 [SMILES: Cc1cc(-c2ccc(N3CCN(Cc4ccc(N5CCNCC5)cc4)CC3)nc2C(F)F)cn(C)c1=O]

The title compound was prepared according to the following scheme

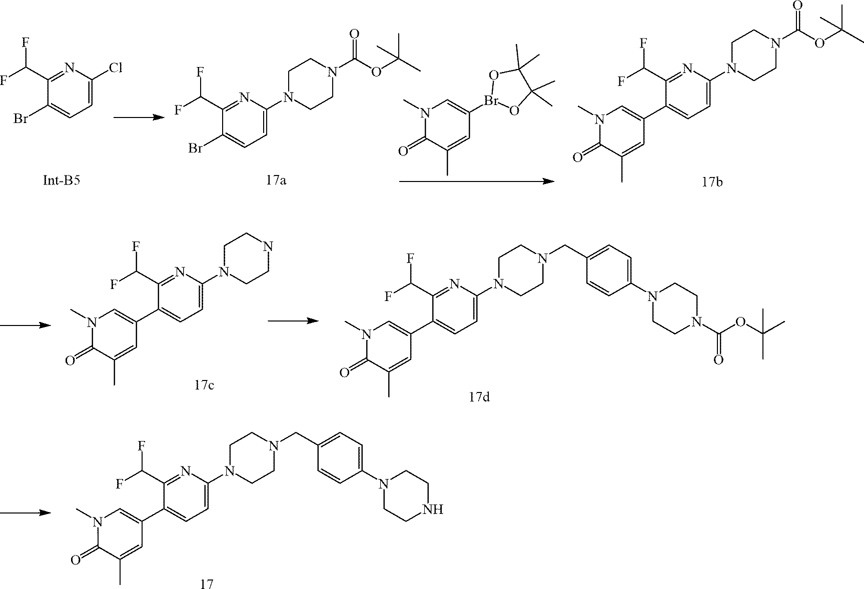

### Step 1: Preparation of Tert-Butyl 4-[5-Bromo-6-(Difluoromethyl)-2-Pyridyl]Piperazine-1-Carboxylate

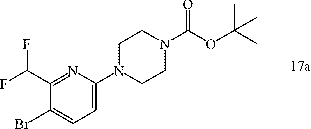 [SMILES: CC(C)(C)OC(=O)N1CCN(c2ccc(Br)c(C(F)F)n2)CC1]

A mixture of Cs₂CO₃ (302 mg, 928 µmol), 3-bromo-6-chloro-2(difluoromethyl)pyridine (150 mg, 619 µmol), tert-butyl piperazine-1-carboxylate (173 mg, 928 µmol)in DMF (3 mL) was stirred at 120° C. for 12 hours. After the reaction was completed, the mixture was diluted with water (10 mL) and the resulting mixture was extracted with DCM (30 mL) twice. The combined organic layer was concentrated in vacuo, the residue was then purified by flash column eluting with a gradient of MeOH/DCM (0% to 10%) to give tert-butyl 4-[5-bromo-6-(difluoromethyl)-2-pyridyl]piperazine-1-carboxylate (123 mg, 50.7% yield) as a yellow oil. MS: calc’d 393 (M+H⁺), measured 393 (M+H⁺).

### Step 2: Preparation of Tert-Butyl 4-[6-(Difluoromethyl)-5-(1,5-Dimethyl-6-Oxo-3-Pyridyl)-2-Pyridyl]Piperazine-1-Carboxylate

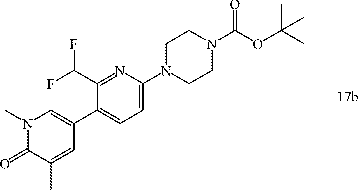 [SMILES: Cc1cc(-c2ccc(N3CCN(C(=O)OC(C)(C)C)CC3)nc2C(F)F)cn(C)c1=O]

To a mixture of tert-butyl 4-(5-bromo-6-(difluoromethyl)pyridin-2-yl)piperazine-1-carboxylate (123 mg, 314 µmol), 1,3-dimethyl-5-(4,4,5,5-tetramethyl-1,3,2-dioxaborolan-2-yl)pyridin-2(1H)-one (78.1 mg, 314 µmol) and K₂CO₃ (65 mg, 470 µmol)in a mixed solvent of dioxane (2 mL) and water (0.5 mL) was added PdCl₂(DPPF)-CH₂Cl₂ adduct (11.5 mg, 15.7 µmol) and the mixture was stirred in dioxane/H₂O (5 : 1, 2.5 mL) at 80° C. under N₂ atmosphere for 16 hr. After the reaction was completed, the mixture was concentrated in vacuo. The residue was then purified by flash column eluting with a gradient of MeOH/DCM (0% to 10%) to give tert-butyl 4-[6-(difluoromethyl)-5-(1,5-dimethyl-6-oxo-3-pyridyl)-2-pyridyl]piperazine-1-carboxylate (120 mg, 88.2% yield) as an orange solid. MS: calc’d 435 (M+H⁺), measured 435 (M+H⁺).

### Step 3: Preparation of 5-[2-(Difluoromethyl)-6-Piperazin-1-yl-3-Pyridyl]-1,3-Dimethyl-Pyridin-2-One

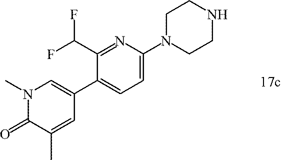 [SMILES: Cc1cc(-c2ccc(N3CCNCC3)nc2C(F)F)cn(C)c1=O]

To a solution of tert-butyl 4-[6-(difluoromethyl)-5-(1,5-dimethyl-6-oxo-3-pyridyl)-2-pyridyl]piperazine-1-carboxylate (120 mg, 275 µmol) in DCM (4 mL) was added (1 mL) and the mixture was stirred at room temperature for 1 hour. After the reaction was completed, the mixture was then concentrated in vacuo, the residue was diluted with 2 M KOH solution (5 mL)and the resulting mixture was extracted with DCM (30 mL) twice. The combined organic layer was concentrated in vacuo to give the crude of 5-[2-(difluoromethyl)-6-piperazin-1-yl-3-pyridyl]-1,3-dimethyl-pyridin-2-one (92.4 mg, 100%) as yellow oil, which was used in the next step directly without further purification. MS: calc’d 335 (M+H⁺), measured 335 (M+H⁺).

### Step 4: Preparation of Tert-Butyl 4-[4-[[4-[6-(Difluoromethyl)-5-(1,5-Dimethyl-6-Oxo-3-Pyridyl)-2-Pyridyl]piperazin-1-yl]Methyl]Phenyl]Piperazine-1-Carboxylate

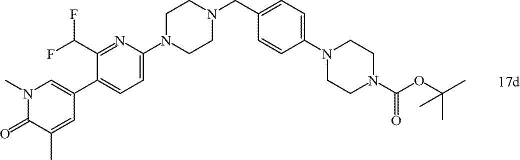 [SMILES: Cc1cc(-c2ccc(N3CCN(Cc4ccc(N5CCN(C(=O)OC(C)(C)C)CC5)cc4)CC3)nc2C(F)F)cn(C)c1=O]

A mixture of 2′-(difluoromethyl)-1,5-dimethyl-6′-(piperazin-1-yl)-[3,3′-bipyridin]-6(1H)-one (92.4 mg, 275 µmol), tert-butyl 4-(4-formylphenyl)piperazine-1-carboxylate (472 mg, 1.62 mmol) and NaBH(OAc)₃ (574 mg, 2.71 mmol) was stirred in DCM (5 mL) at 25° C. for 16 hours. After the reaction was completed, the mixture was then concentrated in vacuo, the residue was then purified by flash column eluting with a gradient of MeOH/DCM (0% to 10%) to give tert-butyl 4-[4-[[4-[6-(difluoromethyl)-5-(1,5-dimethyl-6-oxo-3-pyridyl)-2-pyridyl]piperazin-1-yl]methyl]phenyl]piperazine-1-carboxylate (124 mg, 75.1% yield) as yellow oil. MS: calc’d 609 (M+H⁺), measured 609 (M+H⁺).

### Step 5: Preparation of 5-[2-(Difluoromethyl)-6-[4-[(4-Piperazin-1-Ylphenyl)Methyl] Piperazin-1-yl]-3-Pyridyl]-1,3-Dimethyl-Pyridin-2-One

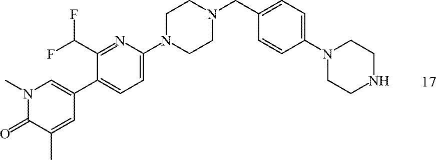 [SMILES: Cc1cc(-c2ccc(N3CCN(Cc4ccc(N5CCNCC5)cc4)CC3)nc2C(F)F)cn(C)c1=O]

To a solution of tert-butyl 4-[4-[[4-[6-(difluoromethyl)-5-(1,5-dimethyl-6-oxo-3-pyridyl)-2-pyridyl]piperazin-1-yl]methyl]phenyl]piperazine-1-carboxylate (125 mg, 205 µmol) in DCM (4 mL) was added TFA (0.5 mL) and the mixture was then stirred at room temperature for 1 hour. After the reaction was completed, the mixture was concentrated in vacuo. The residue was then purified by Prep-HPLC to give 5-[2-(difluoromethyl)-6-[4-[(4-piperazin-1-ylphenyl)methyl]piperazin-1-yl]-3-pyridyl]-1,3-dimethyl-pyridin-2-one (15 mg, 11.5% yield) as a yellow solid. ¹H NMR (400 MHz, CD₃OD, 298 K) δ (ppm) = 7.62 (d, J = 8.7 Hz, 1H), 7.50 -7.43 (m, 3H), 7.38 (s, 1H), 7.16 - 7.07 (m, 3H), 6.75 - 6.41 (m, 1H), 4.77 - 4.39 (m, 2H), 4.33 (s, 2H), 3.68 - 3.33 (m, 14H), 3.31 - 2.99 (m, 3H), 2.15 (s, 3H). ¹⁹F NMR (376 MHz, CD₃OD, 298 K) δ (ppm) = -113.52 (d, J = 54.5 Hz, 2F). MS: calc’d 509 (M+H⁺), measured 509 (M+H⁺).

### Example 18

### 5-Isopropyl-6-(4-Piperazin-1-yl-1-Piperidyl)-3-Pyridyl]-1,3-Dimethyl-Pyridin-2-One

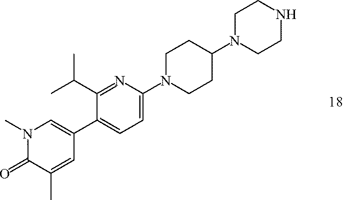 [SMILES: Cc1cc(-c2ccc(N3CCC(N4CCNCC4)CC3)nc2C(C)C)cn(C)c1=O]

The title compound was prepared according to the following scheme

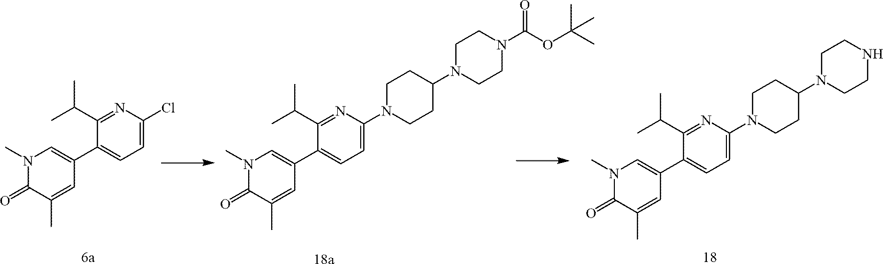

### Step 1: Preparation of Tert-Butyl 4-[1-[5-(1,5-Dimethyl-6-Oxo-3-Pyridyl)-6-Isopropyl-2-Pyridyl]-4-Piperidyl]Piperazine-1-Carboxylate

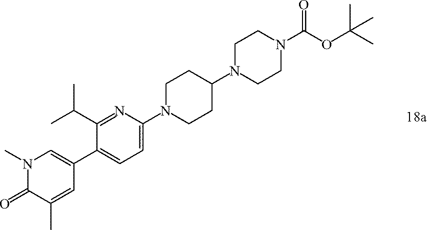 [SMILES: Cc1cc(-c2ccc(N3CCC(N4CCN(C(=O)OC(C)(C)C)CC4)CC3)nc2C(C)C)cn(C)c1=O]

To a mixture of 5-(6-chloro-2-isopropyl-3-pyridyl)-1,3-dimethyl-pyridin-2-one (80 mg, 289 µmol), tert-butyl 4-(4-piperidyl)piperazine-1-carboxylate (100 mg, 371 µmol, CAS No. 205059-24-1, vendor: Bide Pharmatech, catalog BD57121), Cs₂CO₃ (150 mg, 425 µmol) in dioxane (5 mL) was added RuPhos Pd G2 (15 mg, 19.3 µmol) and the mixture was stirred at 110° C. under N₂ atmosphere for 16 hours. After the reaction was completed, the mixture was then concentrated in vacuo. The residue was then purified by flash column eluting with a gradient of MeOH/DCM (0% to 10%) to give tert-butyl 4-[1-[5-(1,5-dimethyl-6-oxo-3-pyridyl)-6-isopropyl-2-pyridyl]-4-piperidyl]piperazine-1-carboxylate (50 mg, 33.9%) as a yellow oil. MS: calc’d 510 (M+H⁺), measured 510 (M+H⁺).

### Step2: Preparation of 5-[2-Isopropyl-6-(4-Piperazin-1-yl-1-Piperidyl)-3-Pyridyl]-1,3-Dimethyl-Pyridin-2-One

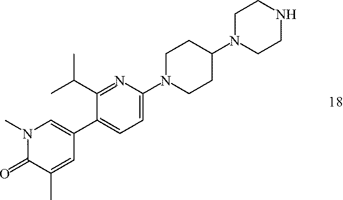 [SMILES: Cc1cc(-c2ccc(N3CCC(N4CCNCC4)CC3)nc2C(C)C)cn(C)c1=O]

To a solution of tert-butyl 4-[1-[5-(1,5-dimethyl-6-oxo-3-pyridyl)-6-isopropyl-2-pyridyl]-4-piperidyl]piperazine-1-carboxylate (50 mg, 0.098 mmol) in DCM (4 mL) was added TFA (1 mL) and the mixture was then stirred at room temperature for 1 hour. After the reaction was completed, the mixture was concentrated in vacuo. The residue was then purified by Prep-HPLC to give 5-[2-isopropyl-6-(4-piperazin-1-yl-1-piperidyl)-3-pyridyl]-1,3-dimethyl-pyridin-2-one (24 mg, 46.7%) as a yellow solid. ¹H NMR (400 MHz, CD₃OD, 298 K) δ (ppm) = 7.44 -7.39 (m, 2H), 7.32 (dd, J = 1.0, 2.3 Hz, 1H), 6.79 (d, J = 8.7 Hz, 1H), 4.59 (br d, J = 13.6 Hz, 2H), 3.61 (s, 3H), 3.59 - 3.50 (m, 8H), 3.49 - 3.41 (m, 1H), 3.15 - 3.06 (m, 1H), 2.99 (br t, J = 11.9 Hz, 2H), 2.24 - 2.12 (m, 5H), 1.76 (br dd, J = 4.0, 12.2 Hz, 2H), 1.20 (d, J = 6.7 Hz, 6H). MS: calc’d 410 (M+H⁺), measured 410 (M+H⁺).

### Example 19

### 5-Ethyl-6-(5-Oxa-2,8-Diazaspiro[3.5]Nonan-2-yl)Pyrimidin-5-yl]-3,4,6,7,9,9a-Hexahydro-1H-Pyrazino[1,2-a]Pyrazin-8-yl]-3-Pyridyl]-1,3-Dimethyl-Pyridin-2-One

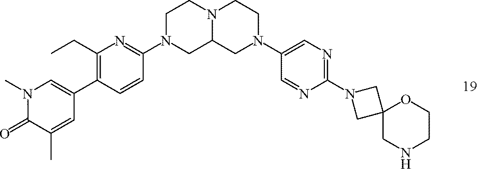 [SMILES: CCc1nc(N2CCN3CCN(c4cnc(N5CC6(CNCCO6)C5)nc4)CC3C2)ccc1-c1cc(C)c(=O)n(C)c1]

The title compound was prepared according to the following scheme

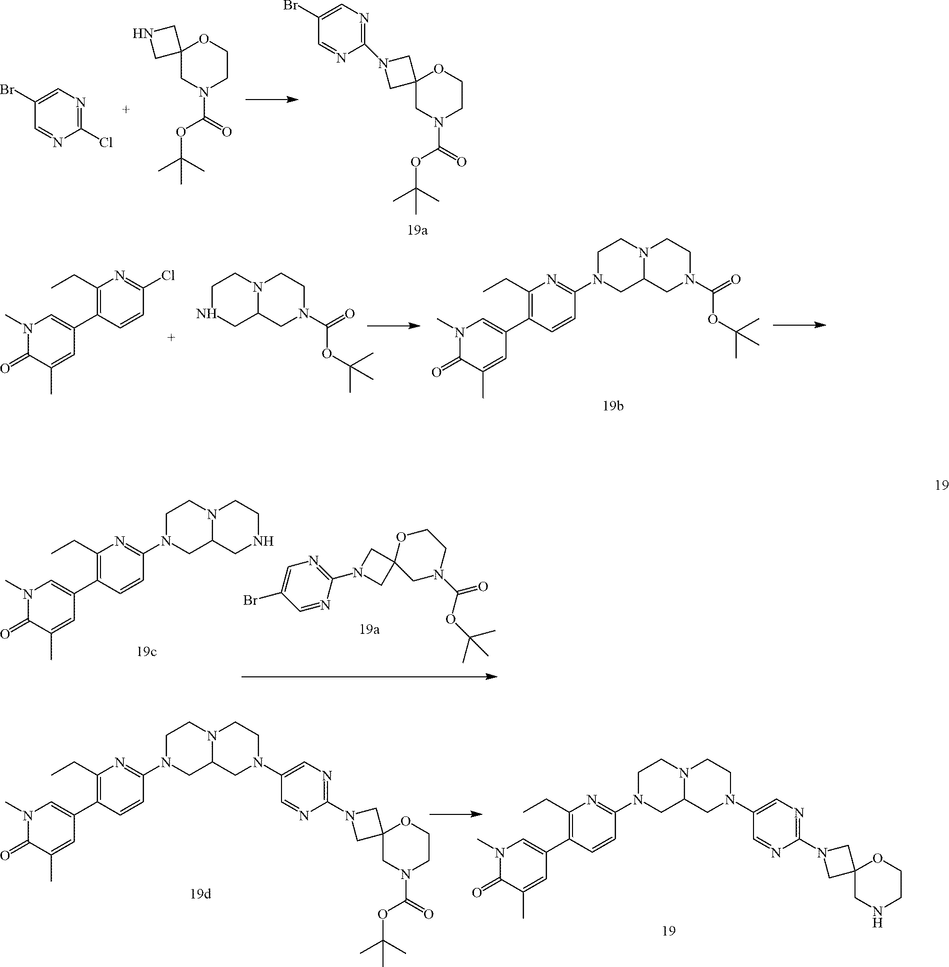

### Step 1: Preparation of Tert-Butyl 2-(5-Bromopyrimidin-2-yl)-5-Oxa-2,8-Diazaspiro[3.5]Nonane-8-Carboxylate

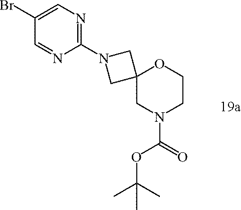 [SMILES: CC(C)(C)OC(=O)N1CCOC2(C1)CN(c1ncc(Br)cn1)C2]

A mixture of 5-bromo-2-chloro-pyrimidine (300 mg, 1.55 mmol, CAS No. 32779-36-5, vendor: Accela ChemBio Inc, catalog 32779-36-5), tert-butyl 5-oxa-2,8-diazaspiro[3.5]nonane-8-carboxylate (425 mg, 1.88 mmol) and K₂CO₃ (322 mg, 2.33 mmol) in DMSO (3 mL) was stirred at 90° C. for 3 hrs. After the reaction was completed, the mixture was diluted with water (5 mL) and the resulting mixture was extracted with DCM (30 mL) twice. The combined organic layer was concentrated in vacuo and the residue was purified by flash column eluting with a gradient of MeOH/DCM (0% to 10%) to give tert-butyl 2-(5-bromopyrimidin-2-yl)-5-oxa-2,8-diazaspiro[3.5]nonane-8-carboxylate (539 mg, 90.2% yield) as a white solid. MS: calc’d 385 (M+H⁺), measured 385 (M+H⁺).

### Step 2: Preparation of Tert-Butyl 8-[5-(1,5-Dimethyl-6-Oxo-3-Pyridyl)-6-Ethyl-2-Pyridyl]-3,4,6,7,9,9a-Hexahydro-1H-Pyrazino[1,2-a]Pyrazine-2-Carboxylate

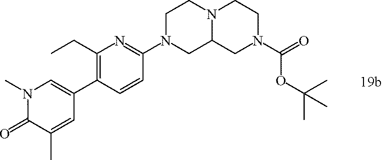 [SMILES: CCc1nc(N2CCN3CCN(C(=O)OC(C)(C)C)CC3C2)ccc1-c1cc(C)c(=O)n(C)c1]

To a mixture of 5-(6-chloro-2-ethyl-3-pyridyl)-1,3-dimethyl-pyridin-2-one (555 mg, 2.1 mmol, compound 1a), tert-butyl 1,3,4,6,7,8,9,9a-octahydropyrazino[1,2-a]pyrazine-2-carboxylate (608 mg, 2.5 mmol, CAS No. 1159825-34-9, vendor: PharmaBlock Sciences (Nanjing), Inc., catalog PB07063) and Cs₂CO₃ (1.0 g, 3.1 mmol) was added RuPhos Pd G2 (81 mg, 110 µmol) and the mixture was stirred in dioxane (5 mL) at 110° C. under N₂ atmosphere for 16 hrs. After the reaction was completed, the mixture was then concentrated in vacuo and the residue was then purified by flash column eluting with a gradient of MeOH/DCM (0% to 10%) to give tert-butyl 8-[5-(1,5-dimethyl-6-oxo-3-pyridyl)-6-ethyl-2-pyridyl]-3,4,6,7,9,9a-hexahydro-1H-pyrazino[1,2-a]pyrazine-2-carboxylate (517 mg, 52.6% yield) as yellow oil. MS: calc’d 468 (M+H⁺), measured 468 (M+H⁺).

### Step 3: Preparation of Preparation of 5-[6-(1,3,4,6,7,8,9,9a-Octahydropyrazino[1,2-a]Pyrazin-2-yl)-2-Ethyl-3-Pyridyl]-1,3-Dimethyl-Pyridin-2-One

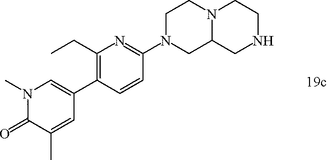 [SMILES: CCc1nc(N2CCN3CCNCC3C2)ccc1-c1cc(C)c(=O)n(C)c1]

To a solution of tert-butyl 8-[5-(1,5-dimethyl-6-oxo-3-pyridyl)-6-ethyl-2-pyridyl]-3,4,6,7,9,9a-hexahydro-1H-pyrazino[1,2-a]pyrazine-2-carboxylate (517 mg, 1.1 mmol) in DCM (4 mL) was added TFA (1 mL) and the mixture was then stirred at room temperature for 1 hour. After the reaction was completed, the mixture was then concentrated in vacuo, the residue was then adjusted to pH~12 by addition of 2 M KOH solution. The resulting mixture was extracted with DCM (30 mL) twice. The combined organic layer was concentrated in vacuo to give the crude of 5-[6-(1,3,4,6,7,8,9,9a-octahydropyrazino[1,2-a]pyrazin-2-yl)-2-ethyl-3-pyridyl]-1,3-dimethyl-pyridin-2-one (376 mg, 93% yield) as orange oil. MS: calc’d 368 (M+H⁺), measured 368 (M+H⁺).

### Step 4: Preparation of Tert-Butyl 2-[5-[8-[5-(1,5-Dimethyl-6-Oxo-3-Pyridyl)-6-Ethyl-2-Pyridyl]-3,4,6,7,9,9a-Hexahydro-1H-Pyrazino[1,2-a]Pyrazin-2-yl]Pyrimidin-2-yl]-5-Oxa-2,8-Diazaspiro[3.5]Nonane-8-Carboxylate

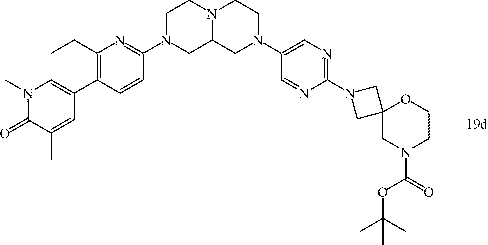 [SMILES: CCc1nc(N2CCN3CCN(c4cnc(N5CC6(CN(C(=O)OC(C)(C)C)CCO6)C5)nc4)CC3C2)ccc1-c1cc(C)c(=O)n(C)c1]

To a mixture of 5-[6-(1,3,4,6,7,8,9,9a-octahydropyrazino[1,2-a]pyrazin-2-yl)-2-ethyl-3-pyridyl]-1,3-dimethyl-pyridin-2-one (200 mg, 0.544 mmol), tert-butyl 2-(5-bromopyrimidin-2-yl)-5-oxa-2,8-diazaspiro[3.5]nonane-8-carboxylate (209.67 mg, 0.544 mmol), Pd₂(dba)₃ (99.67 mg, 0.109 mmol, CAS No. 51364-51-3, vendor: BePharm), 2-(di-t-butylphosphino)biphenyl (64.96 mg, 0.218 mmol, CAS No. 224311-51-7, vendor: J&K Scientific, catalog 912127) in toluene (5 mL) was added sodium tert-butoxide (209.2 mg, 2.18 mmol). The mixture was stirred under N₂ atmosphere at 110° C. for 16 hrs. After the reaction was completed, the mixture was then concentrated in vacuo and the residue was then purified by flash column eluting with a gradient of MeOH/DCM (0% to 15%) to give tert-butyl 2-[5-[8-[5-(1,5-dimethyl-6-oxo-3-pyridyl)-6-ethyl-2-pyridyl]-3,4,6,7,9,9a-hexahydro-1H-pyrazino[1,2-a]pyrazin-2-yl]pyrimidin-2-yl]-5-oxa-2,8-diazaspiro[3.5]nonane-8-carboxylate (90 mg, 24.6% yield) as a yellow oil. MS: calc’d 672 (M+H⁺), measured 672 (M+H⁺).

### Step 5: Preparation of 5-[2-Ethyl-6-[2-[2-(5-Oxa-2,8-Diazaspiro[3.5]Nonan-2-yl)Pyrimidin-5-yl]-3,4,6,7,9,9a-Hexahydro-1H-Pyrazino[1,2-a]Pyrazin-8-yl]-3-Pyridyl]-1,3-Dimethyl-Pyridin-2-One

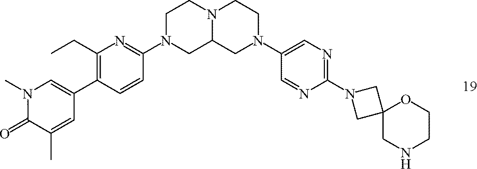 [SMILES: CCc1nc(N2CCN3CCN(c4cnc(N5CC6(CNCCO6)C5)nc4)CC3C2)ccc1-c1cc(C)c(=O)n(C)c1]

To a solution of tert-butyl 2-[5-[8-[5-(1,5-dimethyl-6-oxo-3-pyridyl)-6-ethyl-2-pyridyl]-3,4,6,7,9,9a-hexahydro-1H-pyrazino[1,2-a]pyrazin-2-yl]pyrimidin-2-yl]-5-oxa-2,8-diazaspiro[3.5]nonane-8-carboxylate (90 mg) in DCM (4 mL) was added TFA (1 mL) and the mixture was then stirred at room temperature for 1 hour. After the reaction was completed, the mixture was concentrated in vacuo and the residue was then purified by Prep-HPLC to give 6′-(4-((2-(5-oxa-2,8-diazaspiro[3.5]nonan-2-yl)pyrimidin-5-yl)methyl)piperazin-1-yl)-2′-ethyl-1,5-dimethyl-[3,3′-bipyridin]-6(1H)-one 2,2,2-trifluoroacetate (42 mg) as a yellow solid. ¹H NMR (400 MHz, CD₃OD, 298 K) δ (ppm) = 8.32 (s, 2H), 7.56 (d, J= 8.7 Hz, 1H), 7.48 (d, J= 2.2 Hz, 1H), 7.37 (dd, J = 1.0, 2.4 Hz, 1H), 6.93 (d, J = 8.7 Hz, 1H), 4.63 (br s, 2H), 4.20 (d, J = 10.0 Hz, 2H), 4.08 (d, = 9.8 Hz, 2H), 3.97 (dd, j = 4.1, 5.8 Hz, 2H), 3.82 - 3.74 (m, 1H), 3.73 - 3.63 (m, 4H), 3.63 - 3.60 (m, 3H), 3.52 (s, 2H), 3.47 - 3.33 (m, 3H), 3.30 - 3.17 (m, 4H), 3.14 - 3.03 (m, 1H), 2.74 (q, J = 7.5 Hz, 2H), 2.16 (s, 3H), 1.21 (t, J = 7.5 Hz, 3H). MS: calc’d 572 (M+H⁺), measured 572 (M+H⁺).

### Example 20

The following tests were carried out in order to determine the activity of the compounds of formula (I) and (Ia) in HEK293-Blue-hTLR-7/8/9 cells assay.

### HEK293-Blue-hTLR-7 Cells Assay

A stable HEK293-Blue-hTLR-7 cell line was purchased from InvivoGen (Cat.#: hkb-htlr7, San Diego, California, USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat.#: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000~450,000 cells/mL in a volume of 170 µL in a 96-well plate in Dulbecco’s Modified Eagle’s medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 µL test compound in a serial dilution in the presence of final DMSO at 1% and 10 µL of 20uM R848 in above DMEM, perform incubation under 37° C. in a CO₂ incubator for 20 hrs. Then 20 µL of the supernatant from each well was incubated with 180 µL Quanti-blue substrate solution at 37° C. for 2 hrs and the absorbance was read at 620 \~655 nm using a spectrophotometer. The signaling pathway that TLR7 activation leads to downstream NF-κB activation has been widely accepted, and therefore similar reporter assay was modified for evaluating TLR7 antagonist.

### HEK293-Blue-hTLR-8 Cells Assay

A stable HEK293-Blue-hTLR-8 cell line was purchased from InvivoGen (Cat.#: hkb-htlr8, San Diego, California, USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat.#: rep-qb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000~450,000 cells/mL in a volume of 170 µL in a 96-well plate in Dulbecco’s Modified Eagle’s medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 µL test compound in a serial dilution in the presence of final DMSO at 1% and 10 µL of 60uM R848 in above DMEM, perform incubation under 37° C. in a CO₂ incubator for 20 hrs. Then 20 µL of the supernatant from each well was incubated with 180 µL Quanti-blue substrate solution at 37° C. for 2 hrs and the absorbance was read at 620 \~655 nm using a spectrophotometer. The signaling pathway that TLR8 activation leads to downstream NF-κB activation has been widely accepted, and therefore similar reporter assay was modified for evaluating TLR8 antagonist.

### HEK293-Blue-hTLR-9 Cells Assay

A stable HEK293-Blue-hTLR-9 cell line was purchased from InvivoGen (Cat.#: hkb-htlr9, San Diego, California, USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat.#: tlrl-2006-1, Invivogen, San Diego, California, USA), for incubation of 20 hrs. The cell culture supernatant SEAP reporter activity was determined using QUANTI-Blue™ kit (Cat.#: rep-qb1, Invivogen, San Diego, California, USA) at a wavelength of 640 nm, a detection medium that turns purple or blue in the presence of alkaline phosphatase.

HEK293-Blue-hTLR9 cells were incubated at a density of 250,000~450,000 cells/mL in a volume of 170 µL in a 96-well plate in Dulbecco’s Modified Eagle’s medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 µL test compound in a serial dilution in the presence of final DMSO at 1% and 10 µL of 20uM ODN2006 in above DMEM, perform incubation under 37° C. in a CO₂ incubator for 20 hrs. Then 20 µL of the supernatant from each well was incubated with 180 µL Quanti-blue substrate solution at 37° C. for 2 h and the absorbance was read at 620 \~655 nm using a spectrophotometer. The signaling pathway that TLR9 activation leads to downstream NF-κB activation has been widely accepted, and therefore similar reporter assay was modified for evaluating TLR9 antagonist.

The compounds of formula (I) have human TLR7 and/or TLR8 inhibitory activities (IC₅₀ value) <0.1 µM, TLR9 inhibitory activity <1 µM. Activity data of the compounds of the present invention were shown in Table 1.

TABLE 1

The activity of the compounds of present invention in HEK293-Blue-hTLR-7/8/9 cells assays

| Example No | HEK/hTLR7 IC₅₀ (µM) | HEK/hTLR8 IC₅₀ (µM) | HEK/hTLR9 IC₅₀ (µM) |
|---|---|---|---|
| 1 | 0.009 | 0.0007 | 0.700 |
| 2 | 0.010 | 0.0003 | 0.212 |
| 3 | 0.014 | 0.0003 | 0.621 |
| 4 | 0.024 | 0.0006 | 0.318 |
| 5 | 0.006 | 0.0003 | 0.054 |
| 6 | 0.005 | 0.0003 | 0.198 |
| 7 | 0.005 | 0.0003 | 0.143 |
| 8 | 0.005 | 0.0003 | 0.176 |
| 9 | 0.007 | 0.0003 | 0.183 |
| 10 | 0.007 | 0.0003 | 0.139 |
| 11 | 0.008 | 0.0003 | 0.136 |
| 12 | 0.008 | 0.0007 | 0.763 |
| 13 | 0.012 | 0.0003 | 0.111 |
| 14 | 0.018 | 0.0003 | 0.143 |
| 15 | 0.010 | 0.002 | 0.803 |
| 16 | 0.002 | 0.002 | 0.324 |
| 17 | 0.033 | 0.0003 | 0.068 |
| 18 | 0.005 | 0.0008 | 0.798 |
| 19 | 0.003 | 0.0059 | 0.833 |

### Example 21

### hERG Channel Inhibition Assay

The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells were used for patch-clamp (voltage-clamp, whole-cell) experiments. Cells were stimulated by a voltage pattern to activate hERG channels and conduct I_{KhERG} currents (rapid delayed outward rectifier potassium current of the hERG channel). After the cells were stabilized for a few minutes, the amplitude and kinetics of I_{KhERG} were recorded at a stimulation frequency of 0.1 Hz (6 bpm). Thereafter, the test compound was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%). (references: Redfern WS, Carlsson L, Davis AS, Lynch WG, MacKenzie I, Palethorpe S, Siegl PK, Strang I, Sullivan AT, Wallis R, Camm AJ, Hammond TG. 2003; Relationships between preclinical cardiac electrophysiology, clinical QT interval prolongation and torsade de pointes for a broad range of drugs: evidence for a provisional safety margin in drug development. Cardiovasc. Res. 58:32-45, Sanguinetti MC, Tristani-Firouzi M. 2006; hERG potassium channels and cardiac arrhythmia. Nature 440:463-469, Webster R, Leishman D, Walker D. 2002; Towards a drug concentration effect relationship for QT prolongation and torsades de pointes. Curr. Opin. Drug Discov. Devel. 5:116-26).

Results of hERG are given in Table 2. A safety ratio (hERG IC₂₀ /EC₅₀) > 30 suggests a sufficient window to differentiate the pharmacology by inhibiting TLR7/8/9 pathways from the potential hERG related cardiotoxicity. According to the calculation of hERG IC₂₀ / TLR7/8/9 IC₅₀ below which serves as early selectivity index to assess hERG liability, obviously reference compounds ER-887258, ER-888285, ER-888286, R1 and R2 have much narrower safety window compared to the compounds of this invention.

TABLE 2

hERG and safety ratio results

| Example No | hERG IC₂₀ (µM) | hERG IC₅₀ (µM) | hERG IC₂₀ /TLR7 IC₅₀ | hERG IC₂₀ / TLR8 IC₅₀ | hERG IC₂₀ / TLR9 IC₅₀ |
|---|---|---|---|---|---|
| 1 | >10 | >20.0 | >1052.6 | >14285.2 | >14.2 |
| 2 | >10 | >20.0 | >961.8 | >33000 | >47.1 |
| 3 | >10 | >20.0 | >724.6 | >33000 | >31.4 |
| 7 | 4.7 | >20.0 | 940 | 16000 | 24.4 |

### Example 22

### Human PBMC Cell-Based Assay

Unlike the HEK reporter cell lines, human peripheral blood mononuclear cell (PBMC) represents primary human immune cells in blood mainly consisting of lymphocytes, monocytes, and dendritic cells. These cells express TLR7, TLR8, or TLR9, and therefore are natural responders to respective ligand stimulation. Upon activation of these TLRs, PBMCs secrete similar cytokines and chemokines in vitro and in vivo, and therefore the in vitro potency of a TLR7/8/9 antagonist in human PBMC is readily translatable to its pharmacodynamics response in vivo.

Human peripheral blood mononuclear cells (PBMC) were isolated from freshly-drawn lithium-heparinized (Lithium Heparin Plus blood Collection tube, BD Vacutainer®) healthy donor whole blood by density gradient (Ficoll-PaqueTM PLUS, GE Healthcare life Sciences). Briefly, 50 mL of blood was diluted with 25 mL PBS (without Ca²⁺, Mg²⁺) in a 50 mL conical tube with porous barrier (Leucosep tube, Greiner bio-one), where 15.5 mL Ficoll-Paque was under laid after spinning. Tubes were centrifuged for 20 minutes at 800×g (1946 rpm) with the brake in the off position, and PBMC were collected from the buffy coat. Cells were then washed twice in PBS, and red blood cells were lysed by suspension in 2 mL (Red Blood Cell Lysis Buffer, Alfa Aesar) for 5-10 minutes at room temperature. After a final wash in PBS, PBMC were resuspended at a final concentration of 2×10⁶ cells/mL in RPMI-1640 media with GlutaMAXTM (Gibco) supplemented with 10% Fetal Bovine Serum (Sigma) and plated at 150µL/well (3×10⁵ cells/well) in tissue culture treated round bottom 96-well plates (Corning Incorporated). Antagonist compounds (compounds of this invention) solubilized and serial diluted in 100% DMSO were added in duplicate to cells to yield a final concentration of 1% DMSO (v/v). PBMC were incubated with antagonist compounds for 30 minutes at 37° C., 5% CO₂ before adding various TLR agonist reagents in 48 µL complete media per well as follows (final concentrations indicated): CpG ODN 2216 (InvivoGen) at 1 µM for TLR9, ORN 06/LyoVec (InvivoGen) at 1 µg/mL for TLR8 and R848 (InvivoGen) at 1 µg/mL for TLR7 and TLR8. PBMC were incubated overnight at 37° C. with 5% CO₂. Cell culture supernatants were collected, and levels of various human cytokines were assessed by Luminex assay (ProcartaPlexTM Multiplex Immunoassay, Invitrogen) or ELISA procedure according to the manufacturer’s recommended protocol (eBioscience, ThermoFisher Scientific). Viability of the cells was also checked with Cell Viability Assay (CellTiter Glo®Luminescent Cell Viability Assay, Promega).

TABLE 3

hPBMC results

| Example No | hPBMC/TLR9 IC₅₀ (µM) | hPBMC/TLR78 IC₅₀ (µM) |
|---|---|---|
| 1 | 0.388 | |
| 2 | 0.362 | 0.007 |
| 3 | 0.377 | 0.021 |
| 6 | 0.730 | |
| 7 | 0.101 | |
| 9 | 0.047 | 0.0004 |
| 12 | 0.115 | 0.012 |
| 16 | 0.263 | 0.002 |
| 18 | 0.700 | |

### Example 23

### Human Microsome Stability Assay

The human microsomal stability assay is used for early assessment of metabolic stability of a test compound in human liver microsomes.

Human liver microsomes (Cat.NO.: 452117, Corning, USA;Cat.NO.:H2610, Xenotech, USA) were preincubated with test compound for 10 minutes at 37° C. in 100 mM potassium phosphate buffer, pH 7.4. The reactions were initiated by adding NADPH regenerating system. The final incubation mixtures contained 1 µM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 µL of cold acetonitrile (including internal standard) was added to 100 µL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 4 below.

TABLE 4

Human liver microsome stability of the compounds of present invention

| Example No | Clearance of Human microsome (mL/min/kg) |
|---|---|
| 1 | 6.5 |
| 2 | 12.0 |
| 3 | 6.1 |
| 7 | 7.4 |
| 8 | 8.3 |
| 9 | 7.8 |
| 12 | 8.8 |
| 14 | 8.3 |
| 15 | 8.7 |
| 17 | 8.5 |
| 18 | 7.5 |

### Example 24

### 3T3 In Vitro Phototoxicity Assay

Phototoxicity is defined as a toxic response that is elicited after the first exposure of the skin to certain chemicals and subsequent exposure to light, or that is induced similarly by skin irradiation after systemic administration of a chemical substance. The assay used in this study is designed to detect the phototoxic potential of a chemical by using a simple in vitro cytotoxicity assay with Balb/c 3T3 mouse fibroblasts. The principle of this test is a comparison of the cytotoxicity of a chemical when tested with and without exposure to a non-toxic dose of UVA-light. Cytotoxicity is expressed as a dose dependent reduction of the growth rate of cells as determined by uptake of the vital dye Neutral Red one day after treatment.

### 1. Method

### Preparation of Stock Solution and Dosage of Test Item

A small amount of substance was weighed and formulated freshly in DMSO just before the start of the exposure of the cells. This stock solution or appropriate dilutions with DMSO were added to the cell suspensions to obtain the required final concentrations. All solutions were generally prepared in Eppendorf caps and discarded after use.

### Reference Substance

Chlorpromazine (HCL) (Sigma, Batch/Lot No.: 120M1328V), test concentration: 300 µg/mL, Solvent: PBS / 3% DMSO

### Measurement of UV Absorption Spectrum

The absorption spectra as such or with UV-A or with UV-B pre-irradiation were recorded between 240 nm and 400 nm with a Lambda-2 spectral photometer (Perkin Elmer).
UV radiation sources: for UV-A: Sol 500 with filter H1
   Main spectrum: 315-690 nm
   Irradiance: approx. 1.67 mW/cm²
   Radiation dose : approx. 5 J/cm²
for UV-B: Philips TL 20W/12
   Main spectrum: 290-320 nm
   Irradiance: approx. 0.083 mW/cm²
   Radiation dose: approx. 0.05 J/cm²

### Determination of Phototoxicity

For this study the Neutral Red uptake (NRU) assay of Borenfreund and Puerner (Borenfreund, E, Puerner JA. Toxicity determined in vitro by morphological alterations and Neutral Red absorption. Toxicology Lett. 1985; 24:119-124.) modified according to INVITTOX protocol No 78 (ERGATT/FRAME data bank of in vitro techniques in toxicology. INVITTOX PROTOCOL No 78. 3T3 NRU Phototoxicity Assay. March 1994) has been adapted to examine a possible phototoxic potential of the test item. This assay is based on the active uptake of the Neutral Red dye into the lysosomes of cultured murine fibroblasts. Because lysosomal membranes are known to be a site of action of many phototoxic compounds, this assay can provide a measure of potential for phototoxic injury.

### Preparation of Cell Culture

A murine fibroblasts clone A 31 (ATCC no. CCL 163 - passage No. 108) were cultured in 175 cm² tissue culture grade flasks, containing sDMEM (Dulbecco’s Minimal Essential Medium, supplemented with 10% fetal calf serum, 2 mM L-glutamine, 100 units/ml Penicillin and 100 µg/ml streptomycin) at 37° C. in a humidified atmosphere of 6% CO₂. Before cells approach confluence they were removed from flasks by trypsinisation. Prior to use in an assay, the cells were transferred to 96-well microtiter plates at a concentration of 1× 10⁴ cells/well in 100 µl volumes of sDMEM and allowed to attach for 24 h.

### Exposure to Test Item

For incubation with murine fibroblasts, the test item was diluted in PBS / 3% DMSO (detailed concentrations see in results).

Culture medium (Dulbecco’s Modified Eagle Medium(DMEM), GlutaMAX (Gibco Ref 21885-025), 10% Fetal Bovine Serum (FBS) (Gibco Ref 10270-106), 100IU/ml Penicillin and 100 µg/ml Streptomycin (Gibco Ref 15140-122)) was removed from the wells and murine fibroblasts were washed with PBS. Afterwards 100 µL of PBS / 3% DMSO containing the test item was added and target cells were incubated for 1 h at 37° C. with 6% CO₂.

### UV Exposure

For each test item the microtiter plates were prepared according to Table 6. “UVA plates” were exposed to approx. 5 J/cm² UVA light, the “Dark plates” were kept in the dark and served as cytotoxicity control. Plates with chlorpromazine hydrochloride served as positive control. UV flux was measured with a UV-meter (Dr. Gröbel RM21).

Following UV irradiation, the test item was removed from the wells (one washing step with PBS) and replaced with sDMEM. Target cells were then incubated overnight at 37° C. in 6% CO₂.

TABLE 5.

96-well microtiter plate setup

| | | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| A | S1 | S2 | | | | | | | | | S2 | S1 |
| B | S1 | S2 | | | | | | | | | S2 | S1 |
| C | S1 | S2 | | | | | | | | | S2 | S1 |
| D | S1 | S2 | U01 | U02 | U03 | U04 | U05 | U06 | U07 | U08 | S2 | S1 |
| E | S1 | S2 | | | | | | | | | S2 | S1 |
| F | S1 | S2 | | | | | | | | | S2 | S1 |
| G | S1 | S2 | | | | | | | | | S2 | S1 |
| H | S1 | S2 | | | | | | | | | S2 | S1 |

96-well microtiter plates were prepared as follows:

Each plate contained wells with cells and solvent but without test item which were either not incubated with Neutral Red solution (0% standard - S1) or were stained with Neutral Red (100% standard -S2) for calculation of the standard cell viability curve. Wells labeled with U01-U08 contained the different test item concentrations.

### Neutral Red Uptake

The ready to use Neutral Red (NR) staining solution was freshly prepared as follows:
0.4% aqueous stock solution was shielded from light and filtered before use to remove NR crystals.
1:40 dilution of the stock solution was then prepared in sDMEM and added to the cells.

After the incubation the wells to be assayed were filled with 100 µL of the sDMEM containing Neutral Red. The target cells were incubated with the NR for 3 h at 37° C. in 6% CO₂.

### Measurement of Neutral Red Uptake

Unincorporated Neutral Red was removed from the target cells and the wells washed with at least 100 µL of PBS. 150 µL of Neutral Red desorb solution (1% glacial acetic acid, 50% ethanol in aqua bidest) was then added to quantitatively extract the incorporated dye. After at least 10 mins of vigorous shaking of the plates on a microtiter plate shaker until Neutral Red has been extracted from the cells and formed a homogeneous solution, the absorption of the resulting colored solution was measured with a SPECTRAmax PLUS microtiter plate reader (Molecular Devices) at 540 nm.

### Calculation of Cell Viability

Cell viability was calculated with the SOFTmax Pro software package (Molecular Devices). First a two-point standard curve (0% and 100% viability) was calculated with the linear curve fit option of the program based on the following formula:

$$\text{Y = A +} \left(\text{B} \times \text{X}\right)$$

(A = y-intercept of the line; B = slope of the line;
0% cell viability = cells with solvent, but without test item and Neutral Red;
100% cell viability = cells with solvent and Neutral Red, but without test item)

By this means the viability of the cells incubated with increasing concentrations of the test chemical was calculated. Chlorpromazine (HCl) served as positive control in the experiment.

### Calculation of IC₅₀ Values

All calculations were performed with the SOFTmax Pro analysis software package (Molecular Devices - for details see: http://www.mbl.edu/jbpc/files/2014/05/SoftMax-Pro-User-Guide.pdf)

### Calculation of Discrimination Factor for Phototoxicity

For evaluation of phototoxic potential, the IC₅₀ values determined with and without UV exposure were compared.

$$\text{Factor =} {\text{IC}}_{50} \left(\text{-UV}\right) / {\text{IC}}_{50} \left(+ \text{UV}\right)$$

For discrimination between phototoxic and non-phototoxic test chemicals a cut-off factor of >5 was applied (Liebsch M, Spielmann H, Balls M, Brand M, Döring B, Dupuis J, Holzhüter HG, Klecak G, L.Eplattenier H, Lovell W, Maurer T, Moldenhauer F, Moore L, Pape W, Pfannenbecker U, Potthast JM, De Silva O, Steiling W, Willshaw A. First results of the EC/COLIPA Validation Project. In Vitro Phototoxicity Testing. In: In Vitro Skin Toxicology: Irritation, Phototoxicity, Sensitization; Vol. 10. Alternative Methods in Toxicology,-Eds. Rougier A, Maibach HI, Goldberg AM; Mary Ann Liebert Publ.: New York, USA 1994, pp. 243-251).

Test items which are not cytotoxic to murine fibroblasts even at the highest concentrations tested, but show a strong dose dependent decrease in cell viability after UV exposure are considered also phototoxic (Spielmann H, Balls M, Dupuis J, Pape WJW, Pechovitch G, Silva DeO, Holzhütter, HG, Clothier R, Desolle P, Gerberick F, Liebsch M, Lowell WW, Maurer T, Pfannenbecker U, Potthast JM, Csato M, Sladowski D, Steiling W, Brantom P. The international EU/COLIPA in vitro phototoxicity validation study: Results of phase II (blind trial). Part 1: The 3T3 NRU phototoxicity test. Toxicology in Vitro 1998, 12: 305-327).

The test results were shown below, the compounds of this invention showed very good phototoxicity profile.

TABLE 6

The 3T3 test results for the compound of this invention

| | | |
|---|---|---|
| Example No | Phototoxicity factor | IC₅₀ (UV-A) (µg/mL) |

### Example 25

### Parallel Artificial Membrane Permeability Assay (PAMPA)

PAMPA (Parallel Artificial Membrane Permeability Assay) is a first-line permeability screen for drug candidates. This assay mimics the transcellular absorption conditions using an artificial phospholipid membrane and generates a permeability value that can be used for compound ranking and optimization as well as input parameters for in silico models to predict intestinal absorption.

Permeation experiments are carried out in hydrophobic PVDF 96-well microtiter filter plates (MultiScreen Filter Plate, Millipore, #MAIPN4550). Each well is coated with PVDF membrane, which is prepared with 5 µL Dodecane (Sigma, D221104) that contains 1% lecithin (Sigma, P3556-1G).

The typical PAMPA experimental protocol is as follows: The donor plate is placed on a Teflon acceptor plate that has been pre-filled with 150 µL of 100 mM PBS buffer (2.6 g KH₂PO₄ and 18.5 g K₂HPO₄.3H₂O are dissolved in about 1000 mL of ultra-pure water and mixed thoroughly. The pH is adjusted to 7.40 ± 0.05, using either 1 M sodium hydroxide or 1 M hydrochloric acid.) containing 5% DMSO. The filter on the bottom of each acceptor well is filled with 300 µL of 100 mM PBS buffer (2.6 g KH₂PO₄ and 18.5 g K₂HPO₄.3H₂O are dissolved in about 1000 mL of ultra-pure water, mixed thoroughly. The pH was adjusted to 7.40 ± 0.05, using either 1 M sodium hydroxide or 1 M hydrochloric acid.). The resulting sandwich is incubated at room temperature under constant shaking (300 rpm) for 4 hours. The sandwich is then disassembled. Before incubation, spike 20 µL dosing solution and mix with 250 µL PBS and 130 µL quench solution (acetonitrile) as T0 sample. After incubation, collect 270 µL solutions from acceptor chamber, followed by the addition of 130 µL acetonitrile. Collect 20 µL solution from donor chamber and add 250 µL PBS and 130 µL acetonitrile. The concentration of compound in all samples are determined by LC-MS/MS and the equations for determining the permeability (Pe, 10⁻⁶ cm/s ) are as follows.

$$\begin{matrix}{\text{P}}_{\text{e}} = - \text{C} \times ln \left(1 - \frac{\left({\text{V}}_{\text{D}} + {\text{V}}_{\text{R}}\right) \times {\text{C}}_{\text{R}}}{{\text{V}}_{\text{D}} \times {\text{C}}_{\text{D}} + {\text{V}}_{\text{R}} \times {\text{C}}_{\text{R}}}\right) \text{, where} \\ \text{C} = \frac{{\text{V}}_{\text{D}} \times {\text{V}}_{\text{R}}}{\left({\text{V}}_{\text{D}} + {\text{V}}_{\text{R}}\right) \times \text{Area} \times \text{Time}}\end{matrix}$$

$$\% S o l u t i o n R e c o v e r y = \frac{{C}_{R} \times {V}_{R} + {C}_{D} \times {V}_{D}}{{C}_{D} \times {V}_{D}} \times 1 0 0$$

V_{D} is the volume of the donor well; V_{R} is the volume of the acceptor well; Area is the active surface area of membrane; Time is the incubation time (14,400 s in this assay); C_{R} and C_{D} are the concentrations of compound in acceptor and donor solutions, respectively, at the completion of the assay; C₀ is the concentration of compound in donor solution before incubation.

The main readout of the PAMPA assay is the permeability value Pe expressed in 10⁻⁶ cm/s. Secondary readouts determined are the amounts of compound in the donor and acceptor compartments as well as compound retention in the membrane. Depending on the permeation rate and the membrane retention, compounds are classified as “low” (Pe < 0.2 and membrane retention <20%) or “medium & high” (Pe >= 0.2; or Pe < 0.2 and membrane retention >= 20%). Each sample is measured in triplicate; the standard deviation is determined for the permeation constant Pe. No results are displayed when the sample in acceptor and donor solutions reaches equilibrium (no kinetic information), when the reference is precipitated (turbidity measurement), or in case of analytical limitations.

TABLE 7

Parallel Artificial Membrane Permeability Assay (PAMPA)

| Example No | PAMPA Pe (10⁻⁶cm/s) | Membrane Retention (%) | PAMPA category |
|---|---|---|---|
| 1 | 0.570 | -11.520 | medium & high |
| 2 | 0.490 | 10.600 | medium & high |
| 3 | 0.530 | -1.260 | medium & high |
| 4 | 0.750 | 15.690 | medium & high |
| 5 | 1.500 | 18.250 | medium & high |
| 6 | 0.650 | 19.190 | medium & high |
| 7 | 0.740 | 2.530 | medium & high |
| 8 | 0.950 | 21.370 | medium & high |
| 9 | 0.780 | 27.230 | medium & high |
| 10 | 0.600 | 7.250 | medium & high |
| 11 | 0.600 | 24.900 | medium & high |
| 12 | 0.460 | 15.270 | medium & high |
| 13 | 0.460 | 56.550 | medium & high |
| 14 | 0.540 | 38.020 | medium & high |
| 15 | 0.230 | 27.680 | medium & high |
| 16 | 0.240 | 30.770 | medium & high |
| 17 | 1.500 | 50.950 | medium & high |
| 18 | 0.740 | 8.650 | medium & high |
| 19 | 0.870 | 37.320 | medium & high |

### Example 26

### Single Dose Pharmacokinetics (PK) Study in Male Wister-Han Rats

Pharmacokinetic properties of selected compounds were assessed by single dose PK studies in Male Wister-Han Rats (vendor: Beijing Vital River Laboratory Animal Technology Co., Ltd). Briefly, two groups of animals were administered a single dose of respective compound intravenously (IV, bolus) at 2 mg/kg or orally (PO, by gavage) at 10 mg/kg. Blood samples (approximately 150 µL) were collected via Jugular vein at 5 min (only for IV), 15 min, 30 min, 1 h, 2 h, 4 h, 7 h and 24 h post-dose. Blood samples were placed into tubes containing EDTA-K2 anticoagulant and centrifuged at 3000 rpm for 15 min at 4° C. to separate plasma from the samples. After centrifugation, the resulting plasma was transferred to clean tubes for bioanalysis with LC/MS/MS. The pharmacokinetic parameters were calculated using non-compartmental analysis. The volume of distribution (Vss), half-life (T_{½}) and clearance (CL) were obtained based on the plasma concentration-time curve after IV dose. The peak concentration (Cₘₐₓ) was recorded directly from experimental observations after PO dose. The area under the plasma concentration-time curve (AUC₀₋ₗₐₛₜ) was calculated using the linear trapezoidal rule up to the last detectable concentration. The bioavailability (F) was calculated based on the dose normalized AUC₀₋ₗₐₛₜ after IV and PO dose.

The Vss of a drug represents the degree to which a drug is distributed in body tissue rather than the plasma. Vss is directly proportional with the amount of drug distributed into tissue. A higher Vss indicates a greater amount of tissue distribution.

Results of PK parameters following IV and PO administration are given in Table 9.

TABLE 8

PK parameters for the compounds of this invention

| Example No | PO Cₘₐₓ (ng/mL) | PO AUC₀₋ₗₐₛₜ (h×ng/mL) | IV AUC₀₋ₗₐₛₜ (h×ng/mL) | CL (mL/min/kg) | Vss (L/kg) | T_{½} (h) | F (%) |
|---|---|---|---|---|---|---|---|
| 1 | 151 | 1746 | 873 | 37 | 7.4 | 3.78 | 40 |

### Example 27

### Human Cytosolic Aldehyde Oxidase (AO) Substrate Assay

The Human Cytosolic AO Substrate Assay is to assess the metabolic stability of test compound in human liver cytosol with and without selected aldehyde oxidase (AO) inhibitor. Cytosolic incubations were carried out in deep-well 96-well plates. The conversion of test compound and the formation of oxidized metabolite were monitored over a 60 minutes time period. The volume for incubation was 0.4 mL/well and time points were 0.5, 3.5, 6.5, 10, 20, 30, 45 & 60 minutes. The human liver cytosol (1 mg protein/mL, BD UltraPoolTM Human Cytosol) and test compound (1 µM in duplicate) or control compound (i.e. known AO substrates; 1 µM in duplicate) were incubated at 37° C. in a water bath. At each corresponding time point, 120 µL of quenching solution (Hydralazine in acetonitrile, 50 µM, the total organic concentration will be ≤ 1% in the final incubation) was added to stop the reaction, and a 40 µL sample was withdrawn. All sample plates were mixed well and centrifuged at 3220×g for 10-20 minutes, and supernatants were diluted with water or buffers as appropriate for LC/MS/MS analysis.

In order to determine the in vitro elimination rate of the test compounds and control compound, the analyte/internal standard peak area ratios were converted to percentage remaining with the following equation:

$$\% \text{Remaining} = \frac{\text{Peak area ratio of analyte to internal standard at each time point}}{\text{Peak area ratio of analyte to internal standard at t} \text{=} \text{0}} \times 100 \%$$

And half-life (T_{½}) was calculated from a log linear plot of %Remaining versus time. The estimation of the hepatic intrinsic clearance (CLint) in vitro values were calculated from substrate disappearance rate in liver cytosol incubations as follows: CLint (cytosol) = 0.693/half-life/mg cytosol protein per mL.

TABLE 9

AO parameters for the compounds of this invention

| | | | |
|---|---|---|---|
| Example No | CLint (µL/min/mg) | T_{½} (min) | CLint Ratio to Carbazeran |

1. A compound of formula (I), 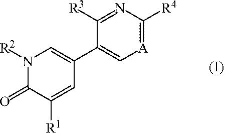
wherein
R¹ is C₁₋₆alkyl;
R² is C₁₋₆alkyl;
R³ is C₁₋₆alkyl or haloC₁₋₆alkyl;
R⁴ is piperazinyl, piperidinyl or 3,4,6,7,9,9a-hexahydro-1H-pyrazino[1,2-a]pyrazinyl, said piperazinyl, piperidinyl or 3,4,6,7,9,9a-hexahydro-1H-pyrazino[1,2-a]pyrazinyl being substituted by substituent selected from
5,6,7,8-tetrahydro-1,6-naphthyridin-2-yl;
phenylC₁₋₆alkyl, wherein phenyl is substituted by piperazinyl;
piperazinyl;
pyrazinylC₁₋₆alkyl, wherein pyrazinyl is substituted by piperazinyl;
pyridinyl, wherein pyridinyl is substituted by piperazinyl;
pyridinylC₁₋₆alkyl, wherein pyridinyl is substituted by 5-oxa-2,8-diazaspiro[3.5]nonan-2-yl or piperazinyl;
pyrimidinyl, where pyrimidinyl is substituted by 5-oxa-2,8-diazaspiro[3.5]nonan-2-yl; and
pyrimidinylC₁₋₆alkyl, wherein pyrimidinyl is substituted by amino(C₁₋₆alkyl)azetidinyl; 5-oxa-2,8-diazaspiro[3.5]nonan-2-yl; amino-1,4-oxazepan-4-yl or piperazinyl;
A is CH or N;
or a pharmaceutically acceptable salt thereof.

2. A compound according to claim 1, wherein A is CH.

3. A compound according to claim 1 or 2, wherein
R⁴ is 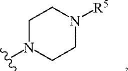 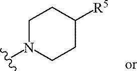 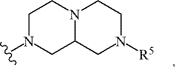
wherein R
⁵ is selected from
5,6,7,8-tetrahydro-1,6-naphthyridin-2-yl;
phenylC₁₋₆alkyl, wherein phenyl is substituted by piperazinyl;
piperazinyl;
pyrazinylC₁₋₆alkyl, wherein pyrazinyl is substituted by piperazinyl;
pyridinyl, wherein pyridinyl is substituted by piperazinyl;
pyridinylC₁₋₆alkyl, wherein pyridinyl is substituted by 5-oxa-2,8-diazaspiro[3.5]nonan-2-yl or piperazinyl;
pyrimidinyl, where pyrimidinyl is substituted by 5-oxa-2,8-diazaspiro[3.5]nonan-2-yl; and
pyrimidinylC₁₋₆alkyl, wherein pyrimidinyl is substituted by amino(C₁₋₆alkyl)azetidinyl; 5-oxa-2,8-diazaspiro[3.5]nonan-2-yl; amino-1,4-oxazepan-4-yl or piperazinyl.

4. A compound according to claim 3, wherein
R⁴ is 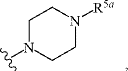
wherein R
^{5a} is 5,6,7,8-tetrahydro-1,6-naphthyridin-2-yl, ((piperazinyl)phenyl)C₁₋₆alkyl, ((piperazinyl)pyrazinyl)C₁-₆alkyl, ((piperazinyl)pyridinyl)C₁₋₆alkyl, ((5-oxa-2,8-diazaspiro[3.5]nonan-2-yl)pyridinyl)C₁₋₆alkyl, ((amino(C₁₋₆alkyl)azetidinyl)pyrimidinyl)C₁₋₆alkyl, ((5-oxa-2,8-diazaspiro[3.5]nonan-2-yl)pyrimidinyl)C₁₋₆alkyl, ((amino-1,4-oxazepan-4-yl)pyrimidinyl)C₁₋₆alkyl or ((piperazinyl)pyrimidinyl)C₁₋₆alkyl; 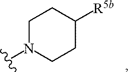
wherein R
^{5b} is piperazinyl; or 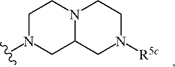
wherein R^{5c} is piperazinylpyridinyl or (5-oxa-2,8-diazaspiro[3.5]nonan-2-yl)pyrimidinyl.

5. A compound according to claim 4, wherein R⁴ is 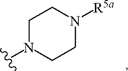
wherein R^{5a} is 5,6,7,8-tetrahydro-1,6-naphthyridin-2-yl, (4-piperazin-1-ylphenyl)methyl, (3-piperazin-1-ylphenyl)methyl, (5-piperazin-1-ylpyrazin-2-yl)methyl, (5-piperazin-1-yl-2-pyridinyl)methyl, (6-piperazin-1-yl-3-pyridinyl)methyl, [6-(5-oxa-2,8-diazaspiro[3.5]nonan-2-yl)-3-pyridinyl]methyl, [2-(3-amino-3-methyl-azetidin-1-yl)pyrimidin-5-yl]methyl, [2-(5-oxa-2,8-diazaspiro[3.5]nonan-2-yl)pyrimidin-5-yl]methyl, [2-[6-amino-1,4-oxazepan-4-yl]pyrimidin-5-yl]methyl, (2-piperazin-1-ylpyrimidin-5-yl)methyl or (5-piperazin-1-ylpyrimidin-2-yl)methyl; 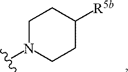
wherein R^{5b} is piperazin-1-yl; or 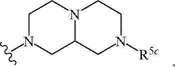
wherein R^{5c} is 6-piperazin-1-yl-3-pyridinyl or 2-(5-oxa-2,8-diazaspiro[3.5]nonan-2-yl)pyrimidin-5-yl.

6. A compound according to claim 4 or 5, wherein R³ is C₁₋₆alkyl.

7. A compound according to claim 6, wherein R³ is ethyl or isopropyl.

8. A compound according to claim 6, wherein R⁴ is 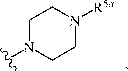
wherein R
^{5a} is ((5-oxa-2,8-diazaspiro[3.5]nonan-2-yl)pyrimidinyl)C₁₋₆alkyl or ((amino-1,4-oxazepan-4-yl)pyrimidinyl)C₁₋₆alkyl;or 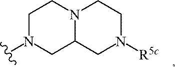
wherein R
^{5c} is (5-oxa-2,8-diazaspiro[3.5]nonan-2-yl)pyrimidinyl.

9. A compound according to claim 8, wherein R⁴ is 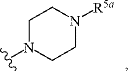
wherein R
^{5a} is [2-(5-oxa-2,8-diazaspiro[3.5]nonan-2-yl)pyrimidin-5-yl] methyl or [2-(3-amino-3-methyl-azetidin-1-yl)pyrimidin-5-yl]methyl; or 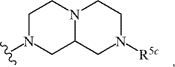
wherein R
^{5c} is 2-(5-oxa-2,8-diazaspiro[3.5]nonan-2-yl)pyrimidin-5-yl.

10. A compound according to claim 1, wherein
R¹ is C₁₋₆alkyl;
R² is C₁₋₆alkyl;
R³ is C₁₋₆alkyl;
R⁴ is 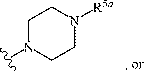
wherein R
^{5a} is ((5-oxa-2,8-diazaspiro[3.5]nonan-2-yl)pyrimidinyl)C₁₋₆alkyl or ((amino-1,4-oxazepan-4-yl)pyrimidinyl)C₁₋₆alkyl; 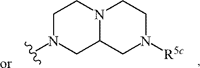
wherein R^{5c} is (5-oxa-2,8-diazaspiro[3.5]nonan-2-yl)pyrimidinyl;
A is CH;
or a pharmaceutically acceptable salt thereof.

11. A compound according to claim 10, wherein
R¹ is methyl;
R² is methyl;
R³ is ethyl or isopropyl;
R⁴ is 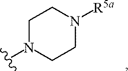
wherein R
^{5a} is [2-(5-oxa-2,8-diazaspiro[3.5]nonan-2-yl)pyrimidin-5-yl]methyl or [2-(3-amino-3-methyl-azetidin-1-yl)pyrimidin-5-yl]methyl; or 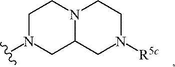
wherein R
^{5c} is 2-(5-oxa-2,8-diazaspiro[3.5]nonan-2-yl)pyrimidin-5-yl;
A is CH;
or a pharmaceutically acceptable salt thereof.

12. A compound selected from:
5-[2-ethyl-6-[4-[[2-(5-oxa-2,8-diazaspiro[3.5]nonan-2-yl)pyrimidin-5-yl]methyl]piperazin-1-yl]-3-pyridyl]-1,3-dimethyl-pyridin-2-one;
5-[2-ethyl-6-[4-[[6-(5-oxa-2,8-diazaspiro[3.5]nonan-2-yl)-3 pyridyl]methyl]piperazin-1-yl]-3-pyridyl]-1,3-dimethyl-pyridin-2-one;
5-[6-[4-[[2-(3-amino-3-methyl-azetidin-1-yl)pyrimidin-5-yl]methyl]piperazin-1-yl]-2-ethyl-3-pyridyl]-1,3-dimethyl-pyridin-2-one;
5-[6-[4-[[2-[(6S)-6-amino-1,4-oxazepan-4-yl]pyrimidin-5-yl]methyl]piperazin-1-yl]-2-ethyl-3-pyridyl]-1,3-dimethyl-pyridin-2-one;
5-[2-ethyl-6-[4-[(4-piperazin-1-ylphenyl)methyl]piperazin-1-yl]-3-pyridyl]-1,3-dimethyl-pyridin-2-one;
5-[2-isopropyl-6-[4-[[2-(5-oxa-2,8-diazaspiro[3.5]nonan-2-yl)pyrimidin-5-yl]methyl] piperazin-1-yl]-3-pyridyl]-1,3-dimethyl-pyridin-2-one;
5-[2-isopropyl-6-[4-[(5-piperazin-1-yl-2-pyridyl)methyl]piperazin-1-yl]-3-pyridyl]-1,3-dimethyl-pyridin-2-one;
5-[2-isopropyl-6-[4-[(5-piperazin-1-ylpyrazin-2-yl)methyl]piperazin-1-yl]-3-pyridyl]-1,3-dimethyl-pyridin-2-one;
5-[2-isopropyl-6-[4-[(2-piperazin-1-ylpyrimidin-5-yl)methyl]piperazin-1-yl]-3-pyridyl]-1,3-dimethyl-pyridin-2-one;
5-[6-[4-[[2-(3-amino-3-methyl-azetidin-1-yl)pyrimidin-5-yl]methyl]piperazin-1-yl]-2-isopropyl-3-pyridyl]-1,3-dimethyl-pyridin-2-one;
5-[2-isopropyl-6-[4-[(6-piperazin-1-yl-3-pyridyl)methyl]piperazin-1-yl]-3-pyridyl]-1,3-dimethyl-pyridin-2-one;
5-[2-isopropyl-6-[4-[(5-piperazin-1-ylpyrimidin-2-yl)methyl]piperazin-1-yl]-3-pyridyl]-1,3-dimethyl-pyridin-2-one;
5-[2-isopropyl-6-[4-[(4-piperazin-1-ylphenyl)methyl]piperazin-1-yl]-3-pyridyl]-1,3-dimethyl-pyridin-2-one;
5-[2-isopropyl-6-[4-[(3-piperazin-1-ylphenyl)methyl]piperazin-1-yl]-3-pyridyl]-1,3-dimethyl-pyridin-2-one;
5-[2-isopropyl-6-[4-(5,6,7,8-tetrahydro-1,6-naphthyridin-2-yl)piperazin-1-yl]-3-pyridyl]-1,3-dimethyl-pyridin-2-one;
5-[2-isopropyl-6-[8-(6-piperazin-1-yl-3-pyridyl)-3,4,6,7,9,9a-hexahydro-1H-pyrazino[1,2-a]pyrazin-2-yl]-3-pyridyl]-1,3-dimethyl-pyridin-2-one;
5-[2-(difluoromethyl)-6-[4-[(4-piperazin-1-ylphenyl)methyl]piperazin-1-yl]-3-pyridyl]-1,3-dimethyl-pyridin-2-one ;
5-[2-isopropyl-6-(4-piperazin-1-yl-1-piperidyl)-3-pyridyl]-1,3-dimethyl-pyridin-2-one; and
5-[2-ethyl-6-[2-[2-(5-oxa-2,8-diazaspiro[3.5]nonan-2-yl)pyrimidin-5-yl]-3,4,6,7,9,9a-hexahydro-1H-pyrazino[1,2-a]pyrazin-8-yl]-3-pyridyl]-1,3-dimethyl-pyridin-2-one;
or a pharmaceutically acceptable salt thereof.

13. A process for the preparation of a compound according to any one of claims 1 to 12 comprising any of the following steps:
a) deprotection of compound of formula (IX), 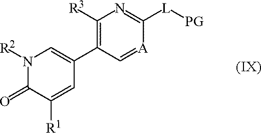
with an acid to afford compound of formula (I-1), 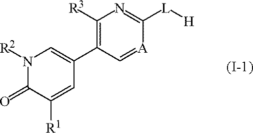
b) deprotection of compound of formula (XV), 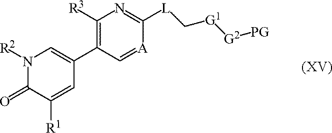
with an acid to afford compound of formula (I-2), 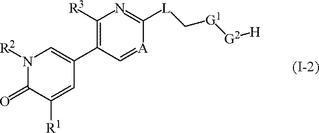
c) deprotection of compound of formula (XVII), 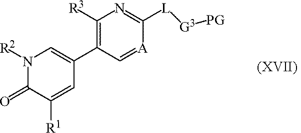
with an acid to afford compound of formula (I-3), 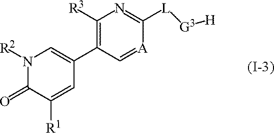
wherein PG is Boc; L is piperazinyl, piperidinyl, piperazinylpiperidinyl, piperidinylpiperazinyl or 3,4,6,7,9,9a-hexahydro-1H-pyrazino[1,2-a]pyrazin-2-yl; G¹ is 5,6,7,8-tetrahydro-1,6-naphthyridin-2-yl, phenyl, piperazinyl, pyrazinyl, pyridinyl or pyrimidinyl; G² is piperazinyl, 5-oxa-2,8-diazaspiro[3.5]nonan-2-yl, amino(C₁₋₆alkyl)azetidinyl or amino-1,4-oxazepan-4-yl; G³ is 5,6,7,8-tetrahydro-1,6-naphthyridin-2-yl; R¹, R², R³ and A are defined as in any one of claims 1 to 11.

14. A compound or pharmaceutically acceptable salt according to any one of claims 1 to 12 for use as therapeutically active substance.

15. A pharmaceutical composition comprising a compound in accordance with any one of claims 1 to 12 and a therapeutically inert carrier.

16. The use of a compound according to any one of claims 1 to 12 for the treatment or prophylaxis of systemic lupus erythematosus or lupus nephritis.

17. The use of a compound according to any one of claims 1 to 12 for the preparation of a medicament for the treatment or prophylaxis of systemic lupus erythematosus or lupus nephritis.

18. The use of a compound according to any one of claims 1 to 12 for the preparation of a medicament for TLR7 and TLR8 and TLR9 antagonist.

19. A compound or pharmaceutically acceptable salt according to any one of claims 1 to 12 for the treatment or prophylaxis of systemic lupus erythematosus or lupus nephritis.

20. A compound or pharmaceutically acceptable salt according to any one of claims 1 to 12, when manufactured according to a process of claim 13.

21. A method for the treatment or prophylaxis of systemic lupus erythematosus or lupus nephritis, which method comprises administering a therapeutically effective amount of a compound as defined in any one of claims 1 to 17.

22. The invention as hereinbefore described.
